(19) United States
(12) Patent Application Publication
(10) Pub. No.: US 2020/0216989 A1
(43) Pub. Date: Jul. 9, 2020

(54) NONWOVEN FABRIC

(71) Applicant: KAO CORPORATION, Tokyo (JP)

(72) Inventors: Yoshihiko KINUGASA, Utsunomiya-shi, Tochigi (JP); Hyuma ITO, Utsunomiya-shi, Tochigi (JP); Masahiro TANIGUCHI, Utsunomiya-shi, Tochigi (JP); Yoshihiko SETO, Utsunomiya-shi, Tochigi (JP); Keiichiro TOMBE, Oyama-shi, Tochigi (JP)

(73) Assignee: KAO CORPORATION, Tokyo (JP)

(21) Appl. No.: 16/628,385

(22) PCT Filed: Jul. 17, 2018
(86) PCT No.: PCT/JP2018/026729

§ 371 Date: Jan. 3, 2020

(30) Foreign Application Priority Data
Aug. 31, 2017 (JP) 2017-168000

(51) Int. Cl. D04H 1/4374; D04H 1/74; D04H 1/76; A61F 13/511; A61F 13/15

(52) U.S. Cl. CPC D04H 1/4374; D04H 1/74; D04H 1/76; A61F 2013/51092; A61F 13/15731; D10B 2401/041; D10B 2509/026; A61F 13/51121

(57) ABSTRACT

A nonwoven fabric, comprising thermoplastic fibers, a first surface side and a second surface side which is a surface side opposite to the first surface side, wherein the nonwoven fabric has outer surface fiber layers on the first surface side and the second surface side in which fibers are oriented in plane direction; and a plurality of connecting portions arranged between the outer surface fiber layer on the first surface side and the outer surface fiber layer on the second surface side, in which fibers are oriented in thickness direction of the nonwoven fabric; and part of the fibers is fused to each other between the outer surface fiber layer on the first surface side, the outer surface fiber layer on the second surface side, and the connecting portions.

### FIELD OF THE INVENTION

The present invention relates to a nonwoven fabric.

### BACKGROUND OF THE INVENTION

Nonwoven fabrics are often used in absorbent articles such as sanitary napkins or diapers. Technology on providing this nonwoven fabric with various functions is known.

Nonwoven fabrics described in Patent Literatures 1 and 2 are directed to processing both fabric sides into concavo-convex surfaces to enhance cushioning properties and the like. The nonwoven fabrics have structures in which a first projecting portion and a second projecting portion projecting in opposite directions to each other are arranged alternately in each of crossing different directions in a plane view through an annular wall portion. Top portions of these projecting portions are formed into a rounded shape for providing a soft texture.

### CITATION LIST

### Patent Literatures

Patent Literature 1: JP-A-2014-12913 (“JP-A” means unexamined published Japanese patent application)

Patent Literature 2: JP-A-2012-136791

### SUMMARY OF THE INVENTION

The present invention provides a nonwoven fabric, comprising thermoplastic fibers, a first surface side and a second surface side which is a surface side opposite to the first surface side, wherein the nonwoven fabric has outer surface fiber layers on the first surface side and the second surface side in which fibers are oriented in plane direction; and a plurality of connecting portions arranged between the outer surface fiber layer on the first surface side and the outer surface fiber layer on the second surface side, in which the fibers are oriented in a thickness direction of the nonwoven fabric; and part of the fibers are fused to each other between the outer surface fiber layer on the first surface side, the outer surface fiber layer on the second surface side, and the connecting portions.

Other and further objects, features and advantages of the invention will appear more fully from the following description, appropriately referring to the accompanying drawings.

### BRIEF DESCRIPTION OF DRAWINGS

FIG. 1 is a perspective view of a partial cross-section schematically showing one preferable embodiment of the nonwoven fabric according to the present invention.

FIG. 2 is a perspective view with partial cutaway schematically showing a specific example when the nonwoven fabric in FIG. 1 is used as a topsheet.

FIG. 3 is a cross-sectional view taken along an A-A line of the nonwoven fabric shown in FIG. 1.

FIG. 4 is a cross-sectional view taken along a B-B line of the nonwoven fabric shown in FIG. 1

FIG. 5 is a drawing substitute photograph showing the measurement of a length of an outer surface fiber layer in a plane direction and a fiber vertical orientation ratio in the cross section of the nonwoven fabric in FIG. 1.

FIG. 6(A) is a plane view showing a first surface side of a nonwoven fabric in the embodiment by partial enlargement of a part, and FIG. 6(B) is a plane view showing a second surface side of the nonwoven fabric in the embodiment by partial enlargement of the part.

FIG. 7 is an explanatory view schematically showing one preferable example of a method for producing a nonwoven fabric in the embodiment, in which FIG. 7(A) is an explanatory view showing a step of arranging a fiber web on a support male material and pushing a support female material into the support male material from above the fiber web, FIG. 7(B) is an explanatory view showing a step of blowing first hot air from above a support female material and shaping the fiber web, and FIG. 7(C) is an explanatory view showing a step of removing the support female material, and blowing second hot air from above a shaped fiber web to fuse fibers to each other.

FIG. 8 is a cross-sectional view showing arrangement of projections of a support male material, projections of a support female material and fibers in a plane direction in which the fibers are planned to be oriented in a thickness direction in the step in FIG. 7(B).

FIG. 9 is a graph showing recoverability after one day of compression of a nonwoven fabric in which sheath-core type conjugate fibers having sheath resin component of polyethylene terephthalate and core resin component of polyethylene are used.

### DESCRIPTION OF EMBODIMENTS

The present invention relates to a nonwoven fabric having sufficient thickness, and simultaneously having a large compressive deformation by compressional load and cushioning properties.

Methods are available for producing a nonwoven fabric having satisfactory texture by providing the nonwoven fabric with cushioning properties. Regarding this point, as a specific example of a method for providing a nonwoven fabric with cushioning properties, the technique of achieving thickness by increasing fiber amount (basis weight) is described. However, increase in the fiber amount is limited in the light of softness and flexibility considerations, as an excessive increase in the fiber amount may adversely affect the texture.

On the other hand, the nonwoven fabrics with concavo-convex surface described in the aforesaid Patent Literature not only have better textures than conventional flat nonwoven fabrics but can also achieve enhanced thickness even if the fiber amount is small. However, there is still room for improvement in the cushioning properties when external force is applied thereto.

The nonwoven fabric according to the present invention secures sufficient thickness, and simultaneously has a large compressive deformation by compressional load and cushioning properties.

A preferred embodiment of the nonwoven fabric according to the present invention will be explained below, referring to the drawings.

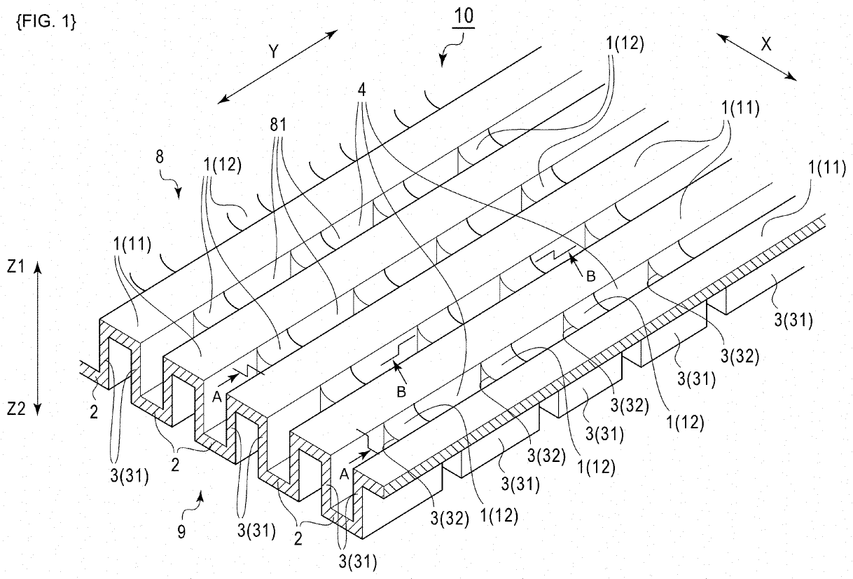

FIG. 1 shows a nonwoven fabric 10 of the embodiment. The nonwoven fabric 10 contains a first surface side Z1, and a second surface side Z2 opposite the first surface side Z1. The first surface side Z1 and the second surface side Z2 mean a top surface side and a back surface side of the nonwoven fabric 10, respectively.

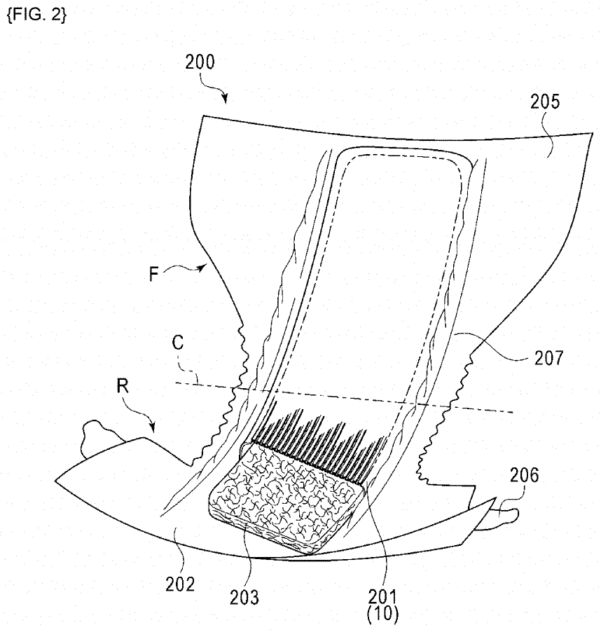

The nonwoven fabric 10 according to the present invention can be applied to a topsheet of an absorbent article such as a sanitary napkin or a disposable diaper, for example. When the nonwoven fabric 10 is used as the topsheet, the nonwoven fabric 10 may be used by directing any surface toward a wearer's skin surface. However, from viewpoints of excellent cushioning properties and soft texture, the first surface side Z1 which is a surface side opposite to a surface to which hot air is blown during production is preferably used toward a wearer's skin surface side because the number of fused points of fibers is comparatively small and the texture is smooth. FIG. 2 shows an example of a diaper 200 in which the nonwoven fabric 10 is arranged as a topsheet 201 by directing the first surface side Z1 toward the wearer's skin surface side. That is, in this example, an outer surface fiber layer 1 on the first surface side Z1 is directed toward the wearer's skin surface side. This diaper 200 has the topsheet 201, and also a backsheet 202 on a clothes side, an absorbent body 203 interposed between the topsheet 201 and the backsheet 202. Further, in the present example, a side leak prevention gather 207 formed by a side sheet is provided. As the diaper 200, a tape-type is shown, in which a fastening tape 206 on a rear side R is fixed on a front side F to wear the diaper 200, but the diaper 200 is not limited thereto, and may be a pants-type diaper. Moreover, the nonwoven fabric can be applied to various absorbent articles other than a diaper, such as a sanitary napkin.

An explanation will now be provided with consideration of an embodiment in which the nonwoven fabric 10 shown in FIG. 1 is used with the first surface side Z1 being toward the skin side. However, the present invention should not be intended to be limited thereto.

The nonwoven fabric 10 of the embodiment contains thermoplastic fibers. The nonwoven fabric 10 is formed by fusion of at least part of the fibers with each other at intersections of the thermoplastic fibers. As elucidated below, the nonwoven fabric 10 has thickness shaped into a form different from conventional sheet-form nonwoven fabrics. Further, the nonwoven fabric 10 exhibits different deformation behavior from conventional sheet-form nonwoven fabrics in a compression direction. This deformation behavior is a load-size dependent behavior, and the nonwoven fabric 10 has characteristic cushioning properties attributable to the behavior. For example, when a low load such as a light finger touch is applied to the nonwoven fabric 10, it is not easily flattened but a resilient force against the finger is produced. This results in a cushion feeling in response to the weak pressure applied by the finger. When a larger load is applied, impact is absorbed by a large compressive deformation, and excellent thickness recoverability is exhibited. Thus, a soft cushion feeling is observed. Namely, the nonwoven fabric 10 has different cushioning properties according to the magnitude of the load.

In addition, (1) good elasticity of the nonwoven fabric when lightly touched, (2) large compressive deformation by compressional load, (3) excellent thickness recoverability, and (4) the presence of cushioning properties can respectively be ascertained by measuring (1) presence or absence of deformation resembling buckling phenomenon (also called buckling distortion), (2) compressive deformation amount, (3) compression recovery rate (RC) and (4) compressive deformation amount and compression energy (WC).

First, the three-dimensional structure of the nonwoven fabric 10 will be described.

The nonwoven fabric 10 has outer surface fiber layers 1 and 2 on the first surface side Z1 and the second surface side Z2, in which the fibers are oriented in a plane direction. In the embodiment, the outer surface fiber layer 1 is present on the first surface side Z1 in a thickness direction Z of the nonwoven fabric 10, and the outer surface fiber layer 2 is present on the second surface side Z2 in the thickness direction Z. Further, between the outer surface fiber layer 1 on the first surface side Z1 and the outer surface fiber layers 2 on the second surface side Z2, is a plurality of connecting portions 3 in which the fibers are oriented in the thickness direction of the nonwoven fabric 10 (hereinafter, the outer surface fiber layer 1 on the first surface side Z1 and the outer surface fiber layer 2 on the second surface side Z2 are also referred to merely as the outer surface fiber layer 1 and the outer surface fiber layer 2.). Between the outer surface fiber layers 1, 2, and the connecting portions 3, at least part of the fibers are fused to each other and seamlessly unified. In the nonwoven fabric 10, the connecting portions 3 support the outer surface fiber layers 1 and 2 by interconnecting them, whereby the nonwoven fabric 10 is formed into a bulky and thick material. Thickness of the nonwoven fabric 10 as termed herein means apparent thickness in a shaped form of the nonwoven fabric as a whole and not local thickness of only the outer surface fiber layers 1, 2 or the connecting portions 3. Specifically in the embodiment, thickness of the nonwoven fabric 10 means thickness between a top surface of the first surface side Z1 and a top surface of the second surface side Z2. This thickness is also referred to as the apparent thickness of the nonwoven fabric 10.

In addition, in the nonwoven fabric 10, the thermoplastic fibers are similarly fused to each other at the intersections of at least part of the fibers at sites other than connection parts between the outer surface fiber layers 1, 2 and the connecting portions 3. The nonwoven fabric 10 may have intersections where the thermoplastic fibers are not fused to each other. Moreover, the nonwoven fabric 10 may contain fibers other than the thermoplastic fibers, and such cases the thermoplastic fibers may be fused at the intersections with the fibers other than thermoplastic fibers.

In the embodiment, the outer surface fiber layer 1 and the outer surface fiber layer 2 are parts in which the fibers are oriented in the plane directions of the first surface side Z1 and the second surface side Z2 respectively of the nonwoven fabric 10.

Here, “fibers are oriented in the plane direction” means that a fiber vertical orientation ratio measured by a method explained later is less than 45%. The fibers can be sufficiently arranged in the plane direction and a flat shape can be maintained by adjusting the fiber vertical orientation ratio to a level less than 45%. With a view of maintaining the shape and strength of the nonwoven fabric, the outer surface fiber layer oriented in the plane direction is preferably adjusted to a fiber vertical orientation ratio of 0% or more, and more preferably to 30% or more. Moreover, the fiber vertical orientation ratios in the outer surface fiber layer 1 and the outer surface fiber layer 2 are preferably adjusted to a level less than 40% because this enables the nonwoven fabric to easily make surface contact with a flat surface as in an ordinary flat nonwoven fabric, and the fiber vertical orientation ratio is preferably adjusted to 38% or less, and further preferably to 37% or less.

As mentioned above, the connecting portions 3 are parts in which fibers are oriented in thickness direction of the nonwoven fabric 10.

Here, “fibers are oriented in thickness direction of the nonwoven fabric” means that the fiber vertical orientation ratio as measured by the method explained later is 60% or more. As the connecting portions 3 have the fiber vertical orientation ratio in this range, it is reasonable to say that the fibers are arranged vertically in the thickness direction of the nonwoven fabric 10.

In the connecting portions 3, the fiber vertical orientation ratio is adjusted to 60% or more, and the connecting portions 3 have parts in which the fibers are partially fused to each other. Thus, the connecting portions 3 stand just like posts to provide the nonwoven fabric 10 with good elasticity in thickness direction. In contrast, the fibers of conventional nonwoven fabrics do not have fiber vertical orientation ratios like that in the connecting portions 3 in the embodiment, so that when a conventional nonwoven fabric is pressed in thickness direction, it deforms so as to fill in interfiber regions with applied force, and the deformation amount increases with force. However, in the embodiment, the connecting portions 3 support the outer surface fiber layers 1 and 2 in the manner of posts, and they extend vertically in thickness direction. The connecting portions 3 can, therefore, withstand slight force applied in the thickness direction. Further, in the embodiment, if large force is applied to the nonwoven fabric 10, the connecting portions 3 deform in a manner that bends the posts. More specifically, deformation occurs that resembles the so-called buckling phenomenon (hereinafter, also referred to as buckling distortion) and that is not observed in the conventional nonwoven fabrics. However, even when connecting portions in the nonwoven fabric 10 are bent in the manner of the buckling phenomenon, the nonwoven fabric 10 can recover its original thickness by its elasticity explained later.

From a viewpoint of the cushioning properties, the fiber vertical orientation ratio in the connecting portions 3 defined as described above is preferably 63% or more, more preferably 65% or more, and further preferably 68% or more. Upper limit thereof is not particularly defined, but in order to establish a force withstanding structure by creating and fusing intersections among the fibers so as to obtain fiber collaborating post, the vertical orientation ratio is preferably 90% or less, more preferably 85% or less, and further preferably 80% or less. Specifically, the vertical orientation ratio in the connecting portions 3 is preferably 63% or more and 90% or less, more preferably 65% or more and 85% or less, and further preferably 68% or more and 80% or less.

The outer surface fiber layers 1, 2 and the connecting portions 3 are parts defined as regions in which the fiber vertical orientation ratio is within the aforesaid range. Ends of the connecting portions 3 are seamlessly connected to the outer surface fiber layers 1 and 2, and fibers oriented in plane direction and fibers oriented in thickness direction are copresent in the end regions. In addition, in parts where fibers oriented in plane direction and fibers oriented in thickness direction are copresent, the fibers are arranged so as to exhibit oblique orientation in which the fiber vertical orientation ratio is preferably 45% or more and 60% or less, and the fiber vertical orientation ratio still more preferably gradually transitions from vertical orientation of 45% to an adequate vertical orientation of 60% or less.

In the nonwoven fabric 10, the outer surface fiber layers 1 and 2 form flat surfaces on both surfaces of the nonwoven fabric 10 by having fiber orientations as described above. Moreover, the connecting portions 3 establish a state in which the connecting portions stand in thickness direction of the nonwoven fabric 10 by having fiber orientation as described above. Moreover, the connecting portions 3 are preferably connected vertically to the outer surface fiber layers 1 and 2 as posts. In particular, taking the cushioning properties described later into consideration, the connecting portions 3 are preferably arranged to link respective end portions of the outer surface fiber layers 1 and 2.

### (Measuring Method of Fiber Vertical Orientation Ratio of Outer Surface Fiber Layer 1, 2 and Connecting Portion 3)

The fiber vertical orientation ratio in the outer surface fiber layers 1, 2 and the connecting portions 3 can be measured based on the following sections (1) to (3).

### (1) Preparation of Cross Section of Nonwoven Fabric

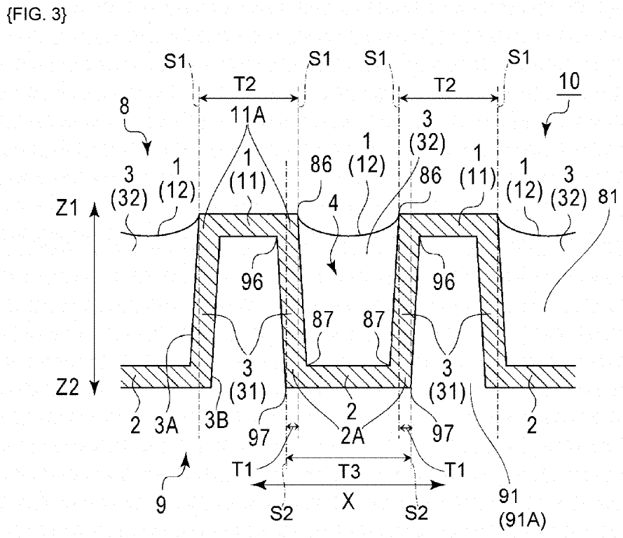

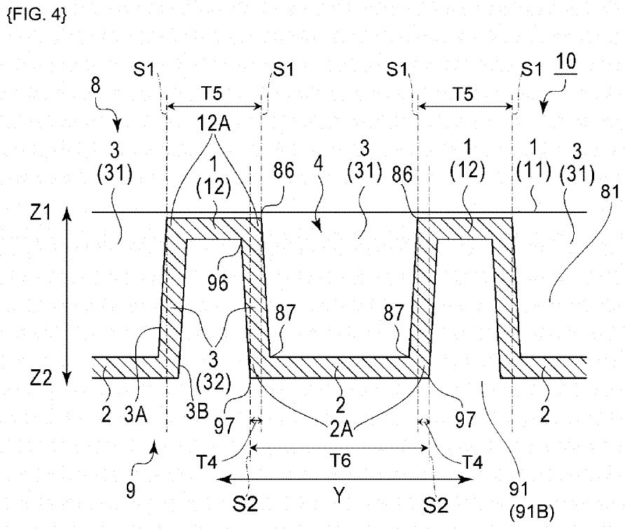

A cross section (vertical cross section) of the nonwoven fabric, which passes through the outer surface fiber layer 1 on the first surface side Z1 and the outer surface fiber layer 2 on the second surface side Z2, and the cross section, in which the connecting portions 3 are perpendicular to a direction extending in plane direction, and in thickness direction in a position passing through a center of the extending length is prepared. Alternatively, when the nonwoven fabric 10 has a space portion 4 as mentioned later, a cross section (vertical cross section) of the nonwoven fabric, which passes through the outer surface fiber layer 1 on the first surface side Z1 and the outer surface fiber layer 2 on the second surface side Z2, and the cross section in thickness direction in the position passing through the center of the space portion 4 is prepared. For example, cross sections (FIGS. 3 and 4) in thickness direction, which pass through an A-A line or a B-B line in FIG. 1, are prepared. The cross section in thickness direction, which passes through the A-A line shown in FIG. 3, is a cross section in which the connecting portions 3 are perpendicular to a longitudinal direction (Y direction) of the nonwoven fabric in which the connecting portions 3 are extended. Here, lengths T1, T2 and T3 of the connecting portions 3, the outer surface fiber layer 1 on the first surface side Z1 and the outer surface fiber layer 2 on the second surface side Z2 in a crosswise direction (X direction) of the nonwoven fabric are shown. The cross section in the thickness direction, which passes through the B-B line shown in FIG. 4, is a cross section in which the connecting portions 3 are perpendicular to the crosswise direction (X direction) of the nonwoven fabric in which the connecting portions 3 are extended. Here, lengths T4, T5 and T6 of the connecting portions 3, the outer surface fiber layer 1 on the first surface side Z1 and the outer surface fiber layer 2 on the second surface side Z2 in the longitudinal direction (Y direction) of the nonwoven fabric are shown. In addition, as the above-described cross section, the nonwoven fabric is cut in a size of 5 mm×5 mm or more.

### (2) Defining of Length of Outer Surface Fiber Layers 1 and 2 in Plane Direction in Cross Section in Thickness Direction

A nonwoven fabric having the above-described cross section in thickness direction is placed on a flat surface, a load of 2.9 Pa is applied onto the nonwoven fabric, and a state is observed from the cross section thereof. Specifically, the nonwoven fabric is put on a baseplate of a digital microscope (VHX-900) manufactured by KEYENCE Corporation. A boundary between the outer surface fiber layers 1 and 2 in the cross section in thickness direction can be judged by placing a black (to facilitate assessment, if the nonwoven fabric is white) cardboard having a basis weight of 300 g/m² on the nonwoven fabric, and observing the nonwoven fabric from the cross section at a magnitude of 20 by using a VHZ20R lens manufactured by KEYENCE Corporation.

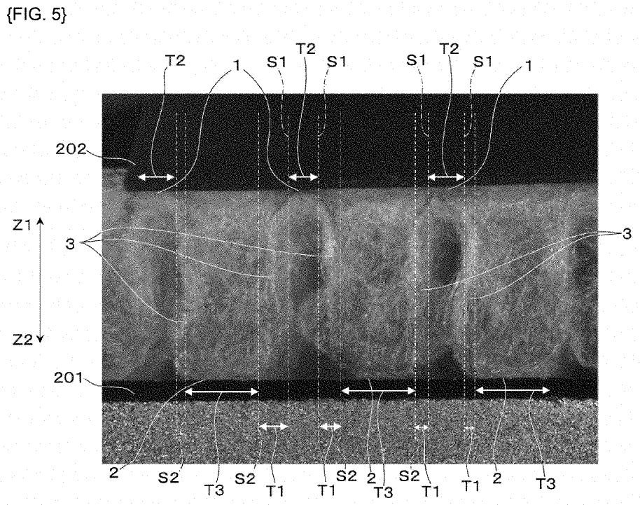

More specifically, in the cross-section observation shown in FIG. 5, a range in contact with a baseplate 201 is defined as a length T3 (or T6) of the outer surface fiber layer 2 in plane direction among the fiber layers showing the cross section in thickness direction, and respective boundaries (both end edges) are specified as S2. A range in contact with a cardboard 202 is defined as a length T2 (or T5) of the outer surface fiber layer 1 in plane direction among the fiber layers processed into the cross section in thickness direction, and respective boundaries (both end edges) are specified as S1. In addition, in the case of the conventional flat nonwoven fabric, if cross-section observation is carried out, the nonwoven fabric is ordinarily brought into contact with the baseplate 201 or the cardboard 202 in any cross section. At this time, a concept of T2 and T3 (or T5 and T6) does not exist.

Moreover, a length T1 (or T4) of the connecting portion 3 in plane direction is determined in the fiber layers showing the cross section in thickness direction. In the embodiment, the connecting portions 3 are arranged in such a manner that the end portions of the outer surface fiber layer 1 on the first surface side Z1 and the outer surface fiber layer 2 on the second surface side Z2 are mutually linked in thickness direction. The length T1 (or T4) of this connecting portion 3 in plane direction is the distance between T2 and T3 (or between T5 and T6) neighbouring to T1 (or T4). More specifically, a length of the connecting portions 3 in plane direction interposed between virtual lines extending, in thickness direction, boundaries (end edges) S1 and S2 of lengths of the outer surface fiber layers 1 and 2 in plane direction is taken as the length T1 (or T4) of the connecting portion 3 in plane direction. In addition, when the length of T1 (or T4) does not exist between T2 and T3 (or between T5 and T6) (that is, when the boundaries S1 and S2 are overlapped), the length of T1 (or T4) is taken as 0. However, as shown in FIG. 4 and FIG. 5, accordingly as the connecting portion 3 approaches arrangement perpendicular to the outer surface fiber layers 1 and 2, the length T1 (or T4) of the connecting portion 3 in plane direction results in a length of a part in which the lengths T2 and T3 (or T5 and T6) of the outer surface fiber layers 1 and 2 in plane direction are partially overlapped.

As the length T1, T2 or T3 (or T4, T5 or T6) in each plane direction is to be defined by cross-section observation, measurement is carried out in four places for each, and an average value thereof is taken as the length.

### (3) Measurement of Fiber Vertical Orientation Ratio in Outer Surface Fiber Layer 1, 2 and Connecting Portions 3

In the fiber vertical orientation ratio in the outer surface fiber layers 1, 2 and the connecting portions 3, measurement is carried out on a site in the range of each of T1, T2 and T3 according to the following procedures.

That is, a cross section in the thickness direction is observed by being magnified to 35 times with SEM (JCM-6000Plus, manufactured by JEOL Ltd.) for each region of a length T2 (or T5) of the outer surface fiber layer 1 in plane direction, a length T3 (or T6) of the outer surface fiber layer 2 in plane direction and a length T1 (or T4) of the connecting portion 3 in plane direction, defined in the cross section in thickness direction. A square of 0.5 mm×0.5 mm is prepared the edges being reference lines for an observed image. Each side (reference line) of the square is taken as a side perpendicular to each in thickness direction and in plane direction in the cross section of the nonwoven fabric. The total number of fibers which pass through the reference line formed of each side of the square is counted. The fibers which pass through the reference line of the square perpendicular to the plane direction of the nonwoven fabric are defined as “number of lateral fibers”, and the fibers which pass through the reference line of the square perpendicular to thickness direction of the nonwoven fabric are defined as “number of vertical fibers.” The vertical orientation ratio is calculated from an equation: (number of vertical fibers)/(number of lateral fibers+number of vertical fibers)×100=vertical orientation ratio (%). Measurement is carried out on 4 points for each, and a value obtained by averaging measured values is taken as a value of the vertical orientation ratio. The outer surface fiber layer and the connecting portion each are cut out, respectively, and measurement is carried out on a cut sample.

The nonwoven fabric 10 in the embodiment has good elasticity and excellent cushioning properties unprecedented as described below by fusion of the outer surface fiber layer 1 on the first surface side Z1 and the outer surface fiber layer 2 on the second surface side Z2 which are plane-oriented, and the connecting portions 3 which are thickness-oriented to each other.

That is, orientation properties of the fibers in the connecting portions 3 produce the nonwoven fabric having high elasticity, as a result the nonwoven fabric is not compressed by force (force less than 100 Pa) at a degree of rubbing, with a finger, in one surface side (for example, first surface side Z1) of the nonwoven fabric 10 owing to rigidity of the fibers. In the nonwoven fabric 10 in the embodiment, respective fibers of this connecting portion 3 in thickness direction and the above-mentioned outer surface fiber layers 1 and 2 in plane direction, and the fibers between the sites are fused to each other to form the surface, and therefore elasticity upon touching the nonwoven fabric is high, and the cushion feeling is by far higher than a cushion feeling in a conventional concavo-convex nonwoven fabric. The nonwoven fabric can be felt to have a soft comfortable and thick feeling for the finger in touch therewith.

Further, if pressing force (force assumed to touch the topsheet of the absorbent article during use, and force of about 2.5 kPa) to push the nonwoven fabric 10 in thickness direction is applied onto one surface side of the nonwoven fabric 10, the pressing force is concentrated in thickness direction rather than being dispersed in plane direction from the vicinity of a force point to easily act thereon. In comparison therewith, in the conventional general concavo-convex nonwoven fabric in which the vertical orientation ratio is low, the force is dispersed in plane direction, and there is a correlation between the deformation and the pressing force as mentioned above, and the good elasticity as in the present invention cannot be obtained. However, in the nonwoven fabric 10 in the embodiment, the connecting portions 3 have a high vertical orientation ratio, and therefore the pressing force is transmitted in a direction along an orientation direction of the fibers in the connecting portions 3. As a result, the pressing force causes deformation (bending) close to the above-mentioned buckling phenomenon in an intermediate position of the connecting portions 3, and not such deformation as collapse of the connecting portions 3 as a whole. Thus, the deformation in which the three-dimensional structure of the nonwoven fabric 10 is flattened (settled) in a flat surface form is avoided, and excellent cushioning properties can be obtained even without increasing the basis weight. Then, the compressive deformation amount in which the nonwoven fabric 10 is compressed becomes larger by concentration of the pressing force in comparison with the conventional nonwoven fabric. Furthermore, such compressive deformation is caused as partial compression in the vicinity of the force point in the nonwoven fabric 10. Specifically, for example, when the nonwoven fabric is pressed with a person's finger, a region in the range of 4 cm² being the substantially same area as a size of the finger including the region and a periphery thereof is compressed in thickness direction, and the deformation is suppressed in other regions, and the thickness is easily kept. Thus, deep compression during a high load is caused in the limited range of the nonwoven fabric 10, the three-dimensional structure of the nonwoven fabric 10 as a whole is kept, and comfortable softness of the nonwoven fabric 10 can be kept. Further, a feeling as being enveloped by a thick nonwoven fabric is obtained in a periphery of the pressing finger. Texture is reputedly felt not only in a ball of the finger but also in the periphery thereof (Transactions of the Virtual Reality Society of Japan Vol. 9, No. 2, 2004, Display of Soft Elastic Object by Simultaneous Control of Fingertip Contact Area and Reaction Force). Therefore, it is considered that the nonwoven fabric is felt to have further satisfactory texture by the feeling in which the whole is enveloped.

In addition thereto, the nonwoven fabric 10 is excellent in thickness recoverability after compressive deformation by vertical orientation properties of the above-mentioned fused fibers in the connecting portions 3. That is, if the compressive deformation by the pressing force is released, original apparent thickness of the nonwoven fabric 10 is recovered by elasticity of the fibers in the connecting portions 3. Thus, even if the nonwoven fabric 10 is repeatedly touched, the cushioning properties are recovered, and sustaining force of the cushioning properties is high. As a result, the nonwoven fabric 10 is formed into a material having elasticity and comfortable texture, in which thickness is easily recovered immediately after the hand is released, even if the nonwoven fabric 10 is touched and once deformed.

In the nonwoven fabric 10, a combination between the outer surface fiber layers 1 and 2 is effective for developing the above-mentioned good elasticity and compressive deformation (buckling distortion) which are provided for the nonwoven fabric 10 by the connecting portions 3. When a top surface side is directly touched, if the nonwoven fabric comprises essentially the connecting portions having the high vertical orientation ratio, the connecting portion is formed into a so-called structure in which the posts are merely aligned. These easily laterally fall over and it is hard to ensure that the force is applied suitably in thickness direction in such a manner as to induce the buckling distortion. However, in the nonwoven fabric 10 of the embodiment, the fibers in the plane direction are connected as bridging, and therefore the pressing force is easily concentrated in thickness direction. That is, the outer surface fiber layers 1 and 2 have the fiber orientation in the plane direction as defined above and are connected with the connecting portions 3 by fusion of the fibers, and thus stress is easily concentrated on the connecting portions 3. For example, when the pressing force is applied from the first surface side Z1, the outer surface fiber layer 1 to which the pressing force is applied most markedly is not excessively deformed, and the stress is transmitted to the connecting portions 3 connected by fusion of the fibers. The pressing force applied to the outer surface fiber layer 1 acts, even if the force causes an eccentric load relative to the orientation direction of the fibers of the connecting portions 3, in such a manner that the connecting portions 3 can preferably induce the buckling distortion. Moreover, the outer surface fiber layer 2 on the second surface side Z2 is not excessively deformed by the pressing force transmitted through the connecting portions 3 to support a root of the connecting portions 3 as a terminating site of the connecting portions 3 connected by thermal fusion of the fibers. Thus, the pressing force applied to one surface of the nonwoven fabric 10 can effectively develop the compressive deformation (buckling distortion) limited to the vicinity of the force point of the pressing force without flattening the three-dimensional structure of the nonwoven fabric 10 as a whole.

Moreover, the outer surface fiber layer oriented in plane direction also improves texture other than the cushion feeling. When the texture is confirmed or the like, a person also performs rubbing operation in addition to pushing operation. In this case, smoother texture is realized by existence of an outer surface having orientation along a rubbing direction. The nonwoven fabric 10 has smoothness obtained by orientation in plane direction and the cushion feeling having buckling in thickness direction, thereby providing a unique feeling. Further, the elasticity of the connecting portions 3 acts on the rubbing force, and thickness (bulkiness) of the nonwoven fabric 10 further facilitates the smooth texture. Furthermore, the comfortable texture obtained by the elasticity can also be simultaneously felt.

The nonwoven fabric 10 is formed into a material having thickness (bulkiness) enough to provide the product with the cushioning properties by the three-dimensional structure formed of the outer surface fiber layers 1, 2 and the connecting portions 3 in thickness direction without increasing fiber amount. Therefore, the nonwoven fabric 10 has higher softness, a smaller fiber amount per unit volume and a larger volume than a material provided with thickness by merely increasing the fiber amount. Therefore, the compressive deformation amount can be increased, the cushion feeling can be felt and the texture is satisfactory. Moreover, the above-mentioned orientation of the fibers, provides the nonwoven fabric 10 with good elasticity excellent texture and cushioning properties by the above-mentioned orientation of the fibers.

In the nonwoven fabric 10, to provide the nonwoven fabric 10 with excellent softness and cushioning properties, the apparent thickness and the basis weight are preferably within the following ranges.

The apparent thickness of the nonwoven fabric is preferably 1.5 mm or more, more preferably 2 mm or more, and further preferably 3 mm or more. Moreover, an upper limit of the apparent thickness is not particularly limited, but when the nonwoven fabric 10 is used as the topsheet of the absorbent article, to provide the topsheet with excellent portability or the like, the apparent thickness is preferably 10 mm or less, more preferably 9 mm or less, and further preferably 8 mm or less.

The basis weight of the nonwoven fabric 10 as a whole having the apparent thickness is preferably 100 g/m² or less, more preferably 60 g/m² or less, and further preferably 40 g/m² or less. Moreover, a lower limit of the basis weight is not particularly limited, but to secure the texture of the nonwoven fabric, the basis weight is preferably 8 g/m² or more, more preferably 10 g/m² or more, and further preferably 15 g/m² or more.

### (Measuring Method of Apparent Thickness and Basis Weight of Nonwoven Fabric 10)

### (1) Measuring Method of Apparent Thickness of Nonwoven Fabric:

A sample of nonwoven fabric is cut to 10 cm×10 cm. When an area of 10 cm×10 cm is unable to be taken, the nonwoven fabric is cut to a largest possible area. A thickness at a load of 50 Pa is measured by using a laser displacement sensor head (ZSLD80, manufactured by OMRON Corporation). Measurement is carried out in three places, and an average value is taken as the apparent thickness of the nonwoven fabric 10.

### (2) Measuring Method of Basis Weight of Nonwoven Fabric:

A sample of nonwoven fabric is cut to 10 cm×10 cm. When an area of 10 cm×10 cm is unable to be taken, the nonwoven fabric is cut to a largest possible area. Weight is measured by using a balance, and a measured value is divided by an area, and the resultant value is taken as basis weight.

(3) In addition, when a commercially available absorbent article is used in measurement of the above-described sections (1) and (2), a sample of nonwoven fabric is peeled carefully by solidifying an adhesive used in the absorbent article by a cooling means such as a cold spray, and the resultant sample is measured. In this case, the adhesive is removed by using an organic solvent. This means is the same as the measurement of all other nonwoven fabrics in the present description.

In the nonwoven fabric 10, from a viewpoint of further effectively developing the above-described action, the connecting portions 3 are preferably provided as described below. That is, as shown in FIG. 3, in a cross section of the nonwoven fabric 10 in thickness direction, a length T1 of the connecting portion 3 in plane direction is preferably formed to be smaller than lengths T2 and T3 of the outer surface fiber layer 1 on the first surface side Z1 and the outer surface fiber layer 2 on the second surface side Z2 in plane direction, respectively. This means that the end edges of the outer surface fiber layer 1 and the outer surface fiber layer 2 are connected to the connecting portions 3, and an inclination of the connecting portions 3 connected therewith is limited within the above-described range and further along thickness direction. Thus, when the outer surface fiber layer 1 or 2 is pushed in thickness direction, the connecting portions 3 are difficult to collapse, and the above-mentioned buckling distortion is further easily and clearly induced, providing the nonwoven fabric 10 with superb cushioning properties.

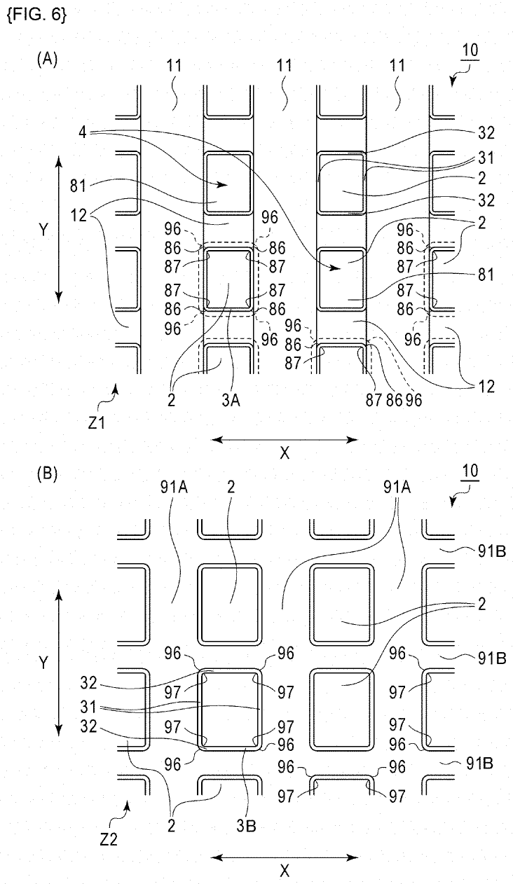

Next, more specific structure of the nonwoven fabric 10 in the embodiment is described referring to FIGS. 1, 3, 4 and 6.

In the embodiment, the outer surface fiber layer 1 on the first surface side Z1 comprises two kinds of parts. The two kinds of parts are a first outer surface fiber layer 11 and a second outer surface fiber layer 12 arranged on the first surface side Z1. These parts each have a length extending along each of crossing in different directions in a plane view of the nonwoven fabric 10. The extending directions are an X direction and a Y direction perpendicular to each other along a side of the nonwoven fabric 10. The Y direction is a longitudinal direction of the nonwoven fabric 10, and the X direction is a crosswise direction of the nonwoven fabric 10.

One first outer surface fiber layer 11 of the two kinds of parts is continuously extended in the Y direction in a plane view of the nonwoven fabric 10, and is continued over the length as a whole of the nonwoven fabric 10. With regard to the first outer surface fiber layers 11 extending in the Y direction, a plurality thereof is separated from each other and arranged relative to the X direction perpendicular to the Y direction.

The other second outer surface fiber layer 12 is extended in the X direction and is arranged by linking the first outer surface fiber layers 11 and 11 separated from each other and arranged in parallel in the X direction. An expression “linking the first outer surface fiber layers 11 and 11” means that the second outer surface fiber layers 12 adjacent to each other by interposing the first outer surface fiber layer 11 are aligned in a linear form. Specifically, the expression means that a deviation of a crosswise center line of the second outer surface fiber layer 12 extending in the X direction from a crosswise center line of the second outer surface fiber layer 12 extending in the X direction, adjacent to each other by interposing the first outer surface fiber layer 11, is within the range of a width (length in the Y direction) of the second outer surface fiber layer 12, for example, within 5 mm. In the second outer surface fiber layer 12, a position on the first surface side Z1 is formed to be somewhat lower than a position of the first outer surface fiber layer 11. Therefore, in the second outer surface fiber layer 12, a length in the X direction is divided by interposition of the first outer surface fiber layer 11, and the plurality thereof form rows in the X direction by being separated from each other. Moreover, a width (width in the Y direction) of the second outer surface fiber layer 12 is formed to be narrower than a width (width in the X direction) of the first outer surface fiber layer 11. With regard to such rows of the second outer surface fiber layers 12 in the X direction, a plurality thereof is further arranged in the Y direction by being separated from each other. In addition, a shape of the second outer surface fiber layer is not limited to a shape in the embodiment, for example, the position and the width on the first surface side Z1 may be adjusted to the same as the position and the width on the first outer surface fiber layer 11. However, spreading of the pressing force in plane direction can be suppressed by adjusting the second outer surface fiber layer 12 to the material in the embodiment, and such a case is preferable.

In addition, when the outer surface fiber layer 1 is provided with a plurality of parts different in the extending directions, as described above, an expression “crossing in different directions in a plane view” deemed as the extending direction is not limited to the X direction and the Y direction. The expression may take various forms as long as the expression refers to crossing directions in the plane direction of the nonwoven fabric 10. From a viewpoint of providing the nonwoven fabric with the superb cushioning properties described above and a viewpoint of ease of preparing vertically oriented fibers in the connecting portion, a crossing angle of “crossing in different directions in a plane view” is most preferably a crossing angle (90°) between a machine direction (MD) of surface fibers in the nonwoven fabric and a cross direction (CD) perpendicular thereto.

With regard to the outer surface fiber layers 2 on the second surface side Z2, a plurality thereof is separated from each other and arranged. Specifically, the outer surface fiber layers 2 on the second surface side Z2 cover separation space between the first outer surface fiber layers 11 and 11 on the first surface side Z1, the plurality thereof are separated from each other and arranged in rows along the extending direction (Y direction) of the outer surface fiber layer 11. Further, with regard to the rows of the outer surface fiber layers 2 in the Y direction, a plurality thereof are separated from each other and arranged in the X direction perpendicular to the Y direction. That is, the outer surface fiber layer 2 is also arrayed in the X direction. Thus, an array direction of the outer surface fiber layers 2 corresponds to the extending direction of the outer surface fiber layer 1. Therefore, when the extending direction of the outer surface fiber layer 1 takes a direction different from the above-described X direction and the above-described Y direction, the array direction of the outer surface fiber layer 2 also takes the direction different from the above-described X direction and the above-described Y direction corresponding thereto.

In addition thereto, the connecting portion 3 contains two kinds of parts. One of the parts is a first connecting portion 31 linking the first outer surface fiber layer 11 on the first surface side Z1 with the outer surface fiber layer 2 on the second surface side Z2 in thickness direction. The other of the parts is a second connecting portion 32 linking the second outer surface fiber layer 12 on the first surface side Z1 with the outer surface fiber layer 2 on the second surface side Z2 in thickness direction. With regard to the connecting portions 3 (the first connecting portions 31 and the second connecting portions 32), a plurality thereof are separated from each other and arranged in plane direction of the nonwoven fabric 10 according to the separation arrangement of the outer surface fiber layers 1 and 2.

The connecting portions 3 have a wall surface having a height in thickness direction of the nonwoven fabric 10, and an extension length (width) in plane direction of the nonwoven fabric 10 along the extending direction of the outer surface fiber layer 1 on the first surface side Z1 and the outer surface fiber layer 2 on the second surface side Z2. The connecting portions 3 link the outer surface fiber layers 1 and 2 by the wall surface, and the wall surfaces are arranged along a plurality of different crossing directions in a plane view in the nonwoven fabric 10. Specifically, the first connecting portions 31 have a length (width) which corresponds to the side of the outer surface fiber layer 2 on the second surface side Z2 in the Y direction and has the wall surface along the extending direction of the first outer surface fiber layer 11 on the first surface side Z1. That is, the wall surface of the first connecting portion 31 is arranged along the Y direction. On the other hand, the second connecting portions 32 have a length (width) which corresponds to the side of the outer surface fiber layer 2 on the second surface side Z2 in the X direction and has the wall surface along the extending direction of the second outer surface fiber layer 12 on the first surface side Z1. That is, the wall surface of the second connecting portion 32 is arranged along the X direction. Thus, a direction along which the wall surface of the connecting portions 3 (first connecting portions 31 and second connecting portions 32) corresponds to the extending of the outer surface fiber layer 1. Therefore, when the extending direction of the outer surface fiber layer 1 takes a direction different from the above-described X direction and the above-described Y direction, the direction along which the wall surface of the connecting portion 3 is arranged also takes a direction different from the above-described X direction and the above-described Y direction corresponding thereto.

The connecting portion 3 links the end portions of the outer surface fiber layer 1 on the first surface side Z1 and the outer surface fiber layer 2 on the second surface side Z2. More specifically, the first connecting portion 31 links an end portion 11A of the first outer surface fiber layer 11 and an end portion 2A of the outer surface fiber layer 2. At this time, as mentioned above with reference to FIG. 3, a length T1 of the first connecting portion 31 in plane direction is preferably smaller than respective lengths T2 and T3 of the first outer surface fiber layer 11 and the outer surface fiber layer 2 in plane direction. On the other hand, the second connecting portion 32 links an end portion 12A of the second outer surface fiber layer 12 and an end portion 2A of the outer surface fiber layer 2. At this time, as shown in FIG. 4 as described above, a length T4 of the second connecting portion 32 in plane direction is preferably smaller than respective lengths T5 and T6 of the second outer surface fiber layer 12 and the outer surface fiber layer 2 in plane direction.

An eccentric load of the pressing force applied to the outer surface fiber layer 1 to the connecting portions 3 becomes clearer by linking the end portions of the outer surface fiber layer 1 and the outer surface fiber layer 2 by the connecting portions 3. At this time, in a combination between the outer surface fiber layers 1 and 2 having fiber orientation in plane direction and the connecting portions 3 having fiber orientation in thickness direction, the load efficiently acts on the end portion, the direction of the pressing force is further concentrated in thickness direction, and deformation behavior easily takes buckling behavior.

Both the first connecting portions 31 and the second connecting portions 32, in which the directions of the wall surfaces are different from each other, are parts in which the fibers are oriented in thickness direction of the nonwoven fabric 10, as defined above. That is, in the connecting portions 3, even if the wall surface is directed in any direction in plane direction of the nonwoven fabric 10 (even if the extending direction is in any direction), the fibers are oriented in thickness direction. The connecting portions 3 directed in a plurality of different directions as described above are unable to be formed in a material in which the fibers are oriented in thickness direction merely by shaping a nonwoven fabric in which the fibers are oriented at random and fused to the concavo-convex surface as in a conventional nonwoven fabric. Even if the fibers are oriented, the fibers are oriented only in one direction of the machine direction (MD) during producing a conventional nonwoven fabric. In contrast, the nonwoven fabric 10 in the embodiment has fiber orientation in thickness direction as defined above also in the connecting portions 3 (connecting portions 31 and 32 having the surfaces perpendicular to each other in the embodiment) in which the wall surfaces are directed in any direction.

Thus, not only when the pressing force is perpendicularly applied, but also when the pressing force is applied in an inclined direction or applied as shear force in multi directions is the above-mentioned buckling distortion in the connecting portions 3 preferably induced to develop excellent cushioning properties in association with the good elasticity of the nonwoven fabric 10.

The nonwoven fabric 10 contains a space portion 4 surrounded by the connecting portions 3 (two first connecting portions 31 and two second connecting portions 32 in the embodiment). The space portion 4 is provided from a region on the first surface side Z1 as divided by the first outer surface fiber layer 11 and the second outer surface fiber layer 12 to a region in thickness direction to the outer surface fiber layer 2 on the second surface side Z2. The space portion 4 has the outer surface fiber layer 2 on the second surface side Z2 as a bottom portion, and is opened on the first surface side Z1. The nonwoven fabric 10 has the space portion 4. Thus, the buckling distortion of the connecting portions 3 is further easily induced, and such a case is preferable. In addition thereto, a compression cushion feeling can be obtained even with weak pressing force (for example, force of about 2.5 kPa in assumption of touching the topsheet of the absorbent article) is applied, and the texture of the nonwoven fabric 10 becomes softer, and such a case is preferable. When the fibers have vertical orientation without any space, the nonwoven fabric becomes harder and the cushion feeling cannot be obtained.

The space portion 4 is formed by being surrounded by four connecting portions 3 vertically arranged from four sides of the outer surface fiber layer 2 on the second surface side Z2. Therefore, with regard to the space portions 4, a plurality thereof is separated from each other and arrayed in corresponding to arrays of the outer surface fiber layer 2 in the X direction and the Y direction. In this array, the space portions 4 are independent without communicating with each other. In the embodiment, a shape formed by the four connecting portions 3 surrounding the space portion 4 and the outer surface fiber layer 2 is formed into a shape of a prismatic body or a frustum. However, the shape of the space portion 4 is not limited thereto and can be formed into various shapes such as a columnar shape, as long as the action mentioned later is produced. In order to disperse a load during touch, a square or a circular cylinder in the bottom surface is more preferable.

All of the connecting portions 3 surrounding the space portion 4 are preferably inclined to the same degree in order to satisfactorily develop the buckling distortion without falling over with the pressing force. That is, in the connecting portions 3, parts in at least four directions surrounding the space portion 4 preferably have the same length in plane direction in the cross section of the nonwoven fabric 10 in thickness direction and the cross section passing through a center of space portion 4. Specifically, the length T1 (FIG. 3) of the first connecting portion 31 in plane direction and the length T4 (FIG. 4) of the second connecting portion 32 in plane direction preferably have the same lengths (T1=T4). Thus, a way of transmitting the pressing force becomes equal also in any connecting portions 3, and even if the pressing force is transmitted from any directions, the buckling distortion may be satisfactorily induced. In addition thereto, in the equalized lengths T1 and T4, a relationship with lengths (T2, T3, T5 and T6) of the outer surface fiber layers 1 and 2 in plane direction are preferably adjusted as mentioned above. Thus, even if the shape formed by the four connecting portions 3 surrounding the space portion 4 and the outer surface fiber layers 2 is has the frustum, the space portion 4 is formed into the shape in which an area difference between upper and lower bottom surfaces in the space portion 4 is minimized. As a result, also in any connecting portions 3 surrounding the space portion 4, the pressing force is easily transmitted along the orientation direction in thickness direction of the fibers, and the buckling distortion is easily induced.

When the length T1 of the first connecting portion 31 in plane direction and the length T4 of the second connecting portion 32 in plane direction are stated to be the same, the statement means that a difference (|T1−T4|) between both is 2 mm or less, and from a viewpoint of improving the buckling distortion of the above-described connecting portion 3, the difference is preferably 1 mm or less, and more preferably 0 (zero) mm.

From the viewpoints of improving the buckling distortion of the above-described connecting portion 3 and further achieving soft texture, a ratio (T1/T2 or T4/T5) of the length (T1 or T4) of the connecting portion 3 in plane direction to the length (T2 or T5) of the outer surface fiber layer 1 on the first surface side Z1 in plane direction is preferably 0.9 or less, more preferably 0.75 or less, and further preferably 0.5 or less. With regard to the ratio (T1/T2 or T4/T5), a smaller ratio is preferable. From the viewpoint of successfully inducing the buckling distortion, the ratio is preferably more than 0, more preferably 0.001 or more, and further preferably 0.01 or more.

Moreover, from viewpoints of stably keeping a nonwoven fabric structure by providing the fibers in parallel to a lower surface and improving the buckling distortion of the above-described connecting portion 3, a ratio (T1/T3 or T4/T6) of the length (T1 or T4) of the connecting portion 3 in plane direction to the length (T3 or T6) of the outer surface fiber layer 2 in plane direction on the second surface side Z2 is also preferably 0.9 or less, more preferably 0.75 or less, and further preferably 0.5 or less in the same manner as the above-mentioned ratio (T1/T2 or T4/T5). With regard to the ratio (T1/T3 or T4/T6), a smaller ratio is preferable. Further, the ratio (T1/T3 or T4/T6) is preferably more than 0, more preferably 0.001 or more, and further preferably 0.01 or more.

Moreover, spreading of the pressing force in plane direction of the nonwoven fabric 10 is suppressed by existence of the space portion 4. Thus, in the nonwoven fabric 10, the compressive deformation (buckling distortion) limited to the narrow region in the vicinity of the force point of the above-mentioned pressing force can be further effectively developed. At this time, the three-dimensional structure of the nonwoven fabric 10 as a whole is easily maintained by existence of the crossing part between the connecting portions 3 surrounding the space portion 4 in combination with the fiber orientation in thickness direction as defined above. Thus, shape recoverability after the buckling distortion is induced in a part other than the crossing part of the connecting portions 3 is high, and the cushioning properties can be further improved.

Further, the space portion 4 is opened on the first surface side Z1. Thus, a body of a person pressing the nonwoven fabric, for example, the skin surface of the finger can be partially entered therein. Thus, when the nonwoven fabric 10 is pressed from the first surface side Z1, together with the compression cushion feeling of the outer surface fiber layer 1 in association with the buckling distortion of the connecting portions 3, a more airy feeling can be obtained in part of the space portion 4, and such a case is preferable. Moreover, if the skin surface of the body is overlapped with the space portion 4, the pressing force is concentrated on the connecting portions 3 in an edge of the space portion 4, and the buckling distortion of the connecting portions 3 is further easily induced. Thus, the cushioning properties of the nonwoven fabric 10 become further preferable. Further, a three-dimensional effect is visually produced by opening, and the texture seems to be psychologically good. Further, upon use as the topsheet of the absorbent article, the opening evokes a region of air permeability to give comfort. Furthermore, the space is kept to form a passage of air, and the air permeability is actually satisfactory to suppress stuffiness.

In the embodiment, a plurality of independent space portions 4 are connected in the Y direction by the first outer surface fiber layer 11 while being separated from each other. Thus, a shape of a surface of the nonwoven fabric 10 on the first surface side Z1 is easily maintained, and shape recoverability after pressing the nonwoven fabric becomes superb, and such a case is preferable. Moreover, heights on the first surface side Z1 are differentiated in the first outer surface fiber layer 11 and the second outer surface fiber layer 12, thereby causing suppression of spreading of the pressing force in plane direction of the nonwoven fabric 10, and such a case is preferable.

In the space portion 4, from a viewpoint of effectively developing the above-described action, an area proportion of the nonwoven fabric 10 in a surface on the first surface side Z1 is preferably 5% or more, more preferably 10% or more, and further preferably 15% or more. Moreover, from a viewpoint of securing strength of the nonwoven fabric, the area proportion of the space portion 4 is preferably 90% or less, more preferably 80% or less, and further preferably 70% or less. Specifically, the area proportion of the space portion 4 is preferably 5% or more and 90% or less, more preferably 10% or more and 80% or less, and further preferably 15% or more and 70% or less.

### (Measuring Method of Area Proportion of Space Portion 4)

A site to be measured from an upper surface is magnified to a size (10 times or more and 100 times or less) which is sufficient for visualisation and can be measured by using a digital microscope (VHX-900) and a VHZ20R lens both manufactured by KEYENCE Corporation, and in the case of also being focused on the lower surface upon being focused on the upper surface, an area of the space portion 4 is measured by taking a fiber region in a place not in focus or a region having no fiber as a space portion, a proportion is calculated from the whole, and taken as an area proportion. When the upper surface and the lower surface are not focused on at the same time, each is focused on, and a region not being focused on each or a region having no fiber is taken as an area proportion.

In the embodiment, the nonwoven fabric 10 has a concavo-convex shape by separation arrangement of the outer surface fiber layer 1 on the first surface side Z1 and the outer surface fiber layer 2 on the second surface side Z2, respectively. This concavo-convex shape has a concavo-convex shape 8 on the first surface side Z1 and a concavo-convex shape 9 on the second surface side Z2. The concavo-convex shape 8 on the first surface side Z1 has a depth equivalent to thickness height of the connecting portion 3 and has a recess portion 81 opened on the first surface side Z1 with the outer surface fiber layer 2 as the bottom portion (see FIGS. 3, 4 and 6(A)). The concavo-convex shape 9 on the second surface side Z2 has a depth equivalent to the thickness height of the connecting portion 3 and has a recess portion 91 which opens on the second surface side Z2 with the outer surface fiber layer 1 as the bottom portion. The recess portion 91 has a recess portion 91A and a recess portion 91B corresponding to two kinds of the outer surface fiber layers 1, respectively (see FIGS. 3, 4 and 6(B)).

The recess portion 91A is in a region on the second surface side Z2 corresponding to the first outer surface fiber layer 11, and has a space between the first connecting portions 31 and 31 along the Y direction (longitudinal direction) of the nonwoven fabric. The recess portion 91A is continued in the Y direction along the extending direction of the first outer surface fiber layer 11.

The recess portion 91B is in a region on the second surface side Z2 corresponding to the second outer surface fiber layer 12, and has a space between the second connecting portions 32 and 32 along the X direction (crosswise direction) of the nonwoven fabric. The recess portion 91B is continued in the X direction along the array direction of the second outer surface fiber layer 12.

The recess portion 91A in the Y direction and the recess portion 91B in the X direction share a space in a crossing part to form a lattice-shaped space on the second surface side Z2 as a whole of the nonwoven fabric 10. Thus, the concavo-convex shapes on the first surface side Z1 and the second surface side Z2 are arranged. The shapes required for buckling are kept by the concavo-convex shapes on the above-described first surface side Z1 and the above-described second surface side Z2, and the texture is improved. Moreover, as mentioned above, deformation further matched with the shape of the finger can be expected by the concavo-convex shape. A person can feel better texture by deformation along the shape of the finger.

Between the outer surface fiber layer 1 and the outer surface fiber layer 2, a fiber amount in one side is preferably adjusted to be smaller than a fiber amount in the other side (namely, a fiber amount in one of the outer surface fiber layer 1 and the outer surface fiber layer 2 is preferably adjusted to be smaller than a fiber amount in the other). Specifically, the fiber amount in the outer surface fiber layer 2 on the second surface side Z2 to which hot air is blown during production is preferably smaller than the fiber amount in the outer surface fiber layer 1 on the first surface side Z1. Thus, a larger amount of fibers is provided on a top surface to be touched, and smooth texture is felt. On the other hand, the fibers on the top surface can be further increased by arranging a minimum amount of fibers capable of keeping the shape on a back surface not to be touched. Further, the fibers do not inhibit absorption to efficiently absorb a liquid by decreasing the fibers on the back surface upon using the nonwoven fabric in the topsheet of the absorbent article. Furthermore, the air permeability can also be improved. These can generate a distribution of fibers by drawing a web before ordinary fusion. If the distribution is desired to be given in upper and lower layers, such a distribution can be achieved by vertically pulling the web. For example, a state in which a larger amount of fibers are distributed upward or downward can be obtained, by vertically pulling the web by interposing the web into intermeshing rolls having concavo-convex shape.

From the viewpoint of improving the above-described action, the fiber amount in the outer surface fiber layer 1 on the first surface side Z1 is preferably 1.1 times or more, more preferably 1.5 times or more, and further preferably 2 times or more than the fiber amount in the outer surface fiber layer 2 on the second surface side Z2. Moreover, from a viewpoint of keeping the shape of the outer surface fiber layer 2, the fiber amount in the outer surface fiber layer 1 on the first surface side Z1 is preferably 20 times or less, more preferably 10 times or less, and further preferably 5 times or less than the fiber amount in the outer surface fiber layer 2 on the second surface side Z2.

### (Measuring Method of Fiber Amount in Outer Surface Fiber Layer 1 and Outer Surface Fiber Layer 2)

A site corresponding to the outer surface fiber layer 1 of the nonwoven fabric and a site corresponding to the outer surface fiber layer 2 thereof are each cut out, mass thereof is measured, measured mass is divided by a cut-out area and the resultant value is taken as the fiber amount (basis weight) (g/m²).

With regard to the outer surface fiber layer 1 and the outer surface fiber layer 2, the number of fused points of the fibers on one surface side is preferably larger than the number thereof on the other surface side. Specifically, the number of fused points of the fibers in the outer surface fiber layer 2 on the second surface side Z2 to which hot air is blown during production is preferably larger than the number thereof in the outer surface fiber layer 1 on the first surface side Z1. Thus, the outer surface fiber layer 2 on the second surface side Z2 has a high absorption of the pressing force to provide the nonwoven fabric 10 as a whole with resilience and to avoid settling. In addition thereto, the shape of the nonwoven fabric 10 is kept by the outer surface fiber layer 2 on the second surface side Z2 having the large number of fused points, thickness increases, and the cushioned feeling is easily felt. Moreover, in the outer surface fiber layer 1 on the first surface side Z1, the number of fused points is small giving smoother texture. In addition, in the embodiment, the surface to which hot air is blown during production is taken as the second surface side Z2, but even if the surface has the same shape as the nonwoven fabric 10, hot air may be blown from the first surface side Z1 to increase the number of fused points between the fibers on the first surface side Z1.

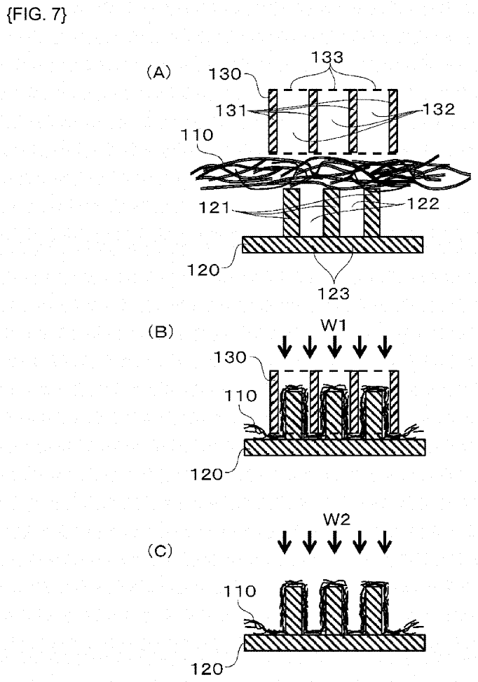

Next, one preferred embodiment of a method for producing the nonwoven fabric 10 of the embodiment is described below, referring to FIG. 7.

In the method for producing the nonwoven fabric 10 in the embodiment, a support male material 120 and a support female material 130 for shaping a fiber web 110 before being processed into the nonwoven fabric are used. As shown in FIG. 7(A), the fiber web 110 is placed on the support male material 120 and pushed, and interposed with the support female material 130 from above to shape the fiber web 110.

The support male material 120 has a plurality of projections 121 corresponding to the four connecting portions 3 surrounding the space portion 4 of the nonwoven fabric 10 and a position in which the outer surface fiber layer 2 on the second surface side Z2 is shaped. A place between the projections 121 and 121 is formed into a recess portion 122 corresponding to the position in which the outer surface fiber layer 1 on the first surface side Z1 is shaped. Thus, the support male material 120 has a concavo-convex shape, and the projections 121 and the recess portions 122 are alternately arranged in different directions in plane view. A bottom portion 123 of the recess portion 122 has a plurality of holes (not shown), through which hot air is blown, for example. In addition, the “different directions” are preferably directions which each correspond to a Y direction (longitudinal direction) and an X direction (crosswise direction) in the nonwoven fabric 10 as a support for producing the nonwoven fabric 10. The Y direction corresponds to a machine direction in the production method, and the X direction corresponds to a crosswise direction perpendicular to the machine direction. However, the “different directions” vary depending on a concavo-convex structure of the nonwoven fabric according to the present invention, and are not limited to the Y direction and the X direction.

The support female material 130 has a lattice-shaped projection 131 corresponding to the recess portions 122 of the support male material 120. A place between the projections 131 and 131 is formed into a recess portion 132 corresponding to the projection 121 of the support male material 120. Thus, the support female material 130 has a concavo-convex shape, and the projections 131 and the recess portions 122 are alternately arranged in different directions in plane view. A bottom portion 133 of the recess portion 132 has a plurality of holes, through which hot air is blown, for example. A distance between the projections 131 and 131 is formed to be wider than a width of the projection 121 of the support male material 120. The distance is appropriately set so as to preferably shape the connecting portions 3 in which the fibers are oriented in thickness direction by interposing the fiber web 110 with the projection 121 of the support male material 120 and the projection 131 of the support female material 130.

First, in the embodiment, the fiber web 110 before being fused is supplied from a carding machine (not shown) to a machine for shaping the web so as to have predetermined thickness.

Next, as shown in FIG. 7(A), the fiber web 110 containing thermoplastic fibers is arranged on the support male material 120, and the support female material 130 is pushed into the support male material 120 from above the fiber web 110. At this time, the projections 121 of the support male materials 120 and the recess portion 132 of the support female material 130 are fitted to each other. Moreover, the recess portions 122 of the support male material 120 and the projections 131 of the support female material 130 are fitted into each other. Thus, a shape in which the fibers are oriented in thickness direction and plane direction is formed.

As shown in FIG. 7(B), in this state, first hot air W1 is blown from a side of the support female material 130 to the fiber web 110. That is, the first hot air W1 is blown from the side serving as a second surface in the nonwoven fabric 10. Thus, in the fiber web 110, the fibers are fused to a degree capable of keeping the concavo-convex shape of the nonwoven fabric 10. The fiber web 110 is formed in a state in which the fibers are significantly loosely fused.

In the fiber web 110, as is different from the nonwoven fabric, the degree of freedom of movement of the fibers is high. Therefore, the fibers are arranged to be easily oriented in the thickness direction (vertical direction) also on a surface toward any direction around the projection 121 of the support male material 120.

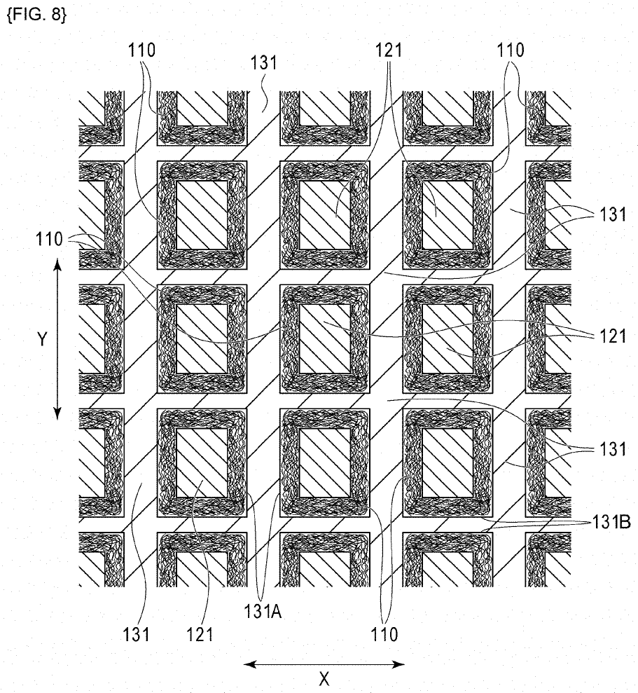

More specifically, if the first hot air W1 is blown from the side of the support female material 130 toward the fiber web 110, the connecting portions 3 of the first nonwoven fabric layer 5 are shaped, in which the fibers are oriented in thickness direction between the wall surface of the projection 121 of the support male material 120 and the wall surface of the projection 131 of the support female material 130. At this time, fusion of intersections between the fibers is not present in the fiber web 110, and therefore mobility of the fibers is high, and orientations of the fibers can be aligned in a blowing direction of the first hot air W1. More specifically, as shown in FIG. 7(B) and FIG. 8, the fibers of the fiber web 110 are aligned in a region interposed between the wall surfaces of the projection 121 of the support male material 120 in all directions, and the wall surface of the projection 131 of the support female material 130 surrounding the wall surfaces. That is, in any of a wall surface 131A of the projection 121 along the machine direction (Y direction) and a wall surface 131B of the projection 121 along the crosswise direction (X direction), the fibers are aligned in the blowing direction of the first hot air W1, irrespective of the direction of the surface. Thus, the fibers in the connecting portions 3 of the nonwoven fabric 10 can be formed, into a configuration in which they are oriented in a thickness direction.

Further, between a top portion of the projection 121 and a bottom portion of the recess portion 132, blowing of the first hot air W1 is suppressed, and the fibers are fused with each other in plane direction. Thus, a fiber layer corresponding to the outer surface fiber layer 2 on the second surface side Z2 is shaped. Moreover, between a bottom portion of the recess portion 122 and a top portion of a protruding portion 131, the fibers are oriented in plane direction. The protruding portion 131 inhibits hot air, and therefore the formed fiber layer has a small amount of fusion, and a smooth fiber layer is obtained. Thus, the fiber layer corresponding to the outer surface fiber layer 1 on the first surface side Z1 is shaped. At this time, a shape of the connecting portions in which the fibers are oriented in thickness direction is also fixed.

In addition, arrows in the drawing schematically show the flow of the first hot air W1.

A temperature of the first hot air W1 is set to a temperature at which the shape of the thermoplastic fibers can be kept in thickness direction and plane direction. The temperature of the first hot air W1 at this time is preferably higher by 0° C. or more and 70° C. or less, more preferably higher by 5° C. or more and 50° C. or less, than the melting point of a thermoplastic fiber constituting the fiber web 110 with consideration of common fiber materials used for products of this type.

From the viewpoint of effectively fusing the fibers, an air speed of the first hot air W1 is preferably 2 m/s or more, and more preferably 3 m/s or more. From the viewpoint of capability of compacting an apparatus scale, the air speed of the first hot air W1 is preferably 100 m/s or less, and more preferably 80 m/s or less.

Thus, the fiber web 110 is temporarily fused to be kept in the concavo-convex shape.

In addition, a height of the projection 121 of the support male material 120 and a height of the projection 131 of the support male material 130 are appropriately determined depending on the apparent thickness of the nonwoven fabric 10 to be produced, or the like. For example, 2 mm or more is preferable, 3 mm or more is preferable, and 5 mm or more is further preferable; and 15 mm or less is preferable, 10 mm or less is more preferable, and 9 or less is further preferable. Specifically, 2 mm or more and 15 mm or less is preferable, 3 mm or more and 10 mm or less is more preferable, and 5 mm or more and 9 mm or less is further preferable.

Next, the support female material 130 is removed, and as shown in FIG. 7(C), second hot air W2 at a temperature at which each fiber in the fiber web 110 shaped in the concavo-convex shape can be properly fused is blown to further fuse the fibers with each other. In the same manner as the first hot air W1 also in this case, the second hot air W2 is blown to the fiber web 110 from the side serving as the second surface in the nonwoven fabric 10. A temperature of the second hot air W2 at this time is preferably higher by 0° C. or more and 70° C. or less, and more preferably higher by 5° C. or more and 50° C. or less, than the melting point of thermoplastic fibers constituting the fiber web 110 with consideration of common fiber materials used for products of this type.

The air speed of the second hot air W2 is, although the setup depends on the height of the projection 121 of the support male material 120, preferably 2 m/s or more, and more preferably 3 m/s or more. Thus, satisfactory heat transfer to the fibers is achieved to fuse the fibers with each other, and satisfactory fixing of the concavo-convex shape can be achieved. Moreover, the air speed of the second hot air W2 is preferably 100 m/s or less, and more preferably 80 m/s or less. Thus, the texture of the nonwoven fabric 10 can be improved by suppressing excessive heat transfer to the fibers.

In addition, a step of blowing the first hot air W1 can be omitted by reducing surface roughness of the support female material. The fibers not fused are not entangled by reducing the surface roughness, and the support female material can be removed in a step of blowing the second hot air W2. That is, the support male material and the support female material are fitted to each other after preparing the web, the support female material is directly removed, and treatment can be applied thereto by the second hot air W2. Thus, simpler processing can be achieved.

As the thermoplastic fibers, the thermoplastic fibers ordinarily used in a raw material of the nonwoven fabric can be adopted without particular restriction. For example, the thermoplastic fibers may be fibers comprising single resin component, conjugate fibers comprising a plurality of resin components, or the like. Specific examples of the conjugate fibers include a sheath-core type and a side-by-side type.

When conjugate fibers comprising a low-melting component and a high-melting component (for example, sheath-core type conjugate fibers in which the sheath is low-melting component and the core is high-melting component) is used as the thermoplastic fibers, a temperature of the hot air to be blown onto the fiber web 110 is preferably equal to or higher than a melting point of the low-melting component and less than a melting point of the high-melting component. The temperature is more preferably equal to or higher than the melting point of the low-melting component and lower by 10° C. than the melting point of the high-melting component, and further preferably higher by 5° C. or more than the melting point of the low-melting component and lower by 20° C. or more than the melting point of the high-melting component. Moreover, from the viewpoint of elasticity, where the elasticity is higher an amount of the core being the high-melting component of sheath-core type conjugate fibers is larger. Therefore, a case where an amount of the core component is larger in a cross-sectional area proportion is preferable. Specific examples of the sheath-core type conjugate fibers in which the sheath is a low-melting component and the core is a high-melting component include sheath-core type conjugate fibers in which the sheath is polyethylene (PE) and the core is polyethylene terephthalate (PET).

Further, in the sheath-core type conjugate fibers, where the sheath resin component has lower glass transition temperature than the core resin component (hereinafter, referred to as low glass transition temperature resin) (for example, the core resin component is PET and the sheath resin component is PE), recoverability of thickness of the nonwoven fabric can be enhanced by reducing the mass ratio of the low glass transition temperature resin component. As factors that contribute to this situation, the following can be considered. It is known that a low glass transition temperature resin has low relaxation modulus. Moreover, it is also known that deformation recovery is reduced/retained when the relaxation modulus is low. Therefore, it is considered that higher thickness recoverability can be provided to the nonwoven fabric by reducing the low glass transition temperature resin component as much as possible.

In the case of the sheath-core type conjugate fibers, a proportion of the low glass transition temperature resin component (PE and the like) with respect to the total fiber mass is preferably smaller than a proportion of the resin component having high glass transition temperature (PET and the like) with respect to the total fiber mass, by mass ratio. Specifically, the proportion of the low glass transition temperature resin component with respect to the total fiber mass is preferably 45 mass % or less, and more preferably 40 mass % or less, by mass ratio. Thickness recoverability of the nonwoven fabric can be enhanced by reducing the proportion of the low glass transition temperature resin component. Moreover, from a viewpoint of producing the nonwoven fabric, the proportion is preferably 10 mass % or more, and more preferably 20 mass % or more, by mass ratio.

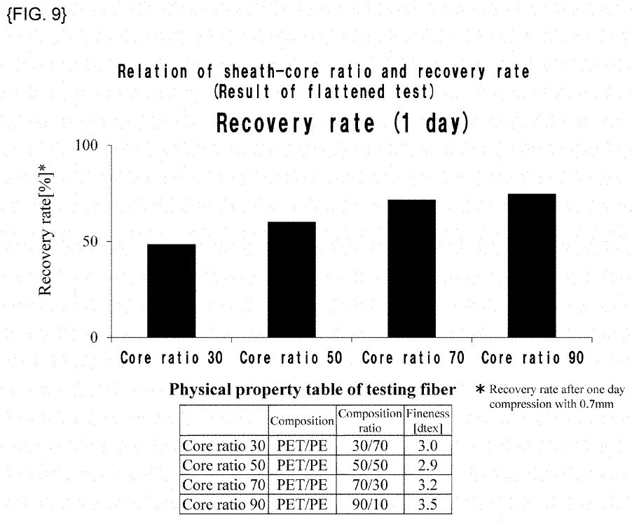

This can also be seen from a graph shown in FIG. 9. FIG. 9 shows recoverability rate after one day of compression of a nonwoven fabric as proportion of the core resin component (PET) and the sheath resin component (PE) is changed (measuring method is based on the method shown in the “(5) Recoverability after one day compression” shown in Examples described below). In addition, this nonwoven fabric was prepared according to an air-through production method including a step shown in FIG. 7. Blowing treatment by first hot air W1 was applied thereto at a temperature of 160° C., an air speed of 54 m/s and a blowing time of 6 s. Blowing treatment by second hot air was applied thereto at a temperature of 160° C., an air speed of 6 m/s and a blowing time of 6 s. The apparent thickness of the prepared nonwoven fabric was 6.0 mm for a type of “core ratio of 30”, 6.9 mm for a type of “core ratio of 50”, 6.6 mm for a type of “core ratio of 70”, and 6.0 mm for a type of “core ratio of 90”. As the proportion of the sheath resin component being PE having low glass transition temperature is smaller (the proportion of the core resin component is larger), the recoverability rate after one day of compression is higher. In particular, when the proportion of the sheath resin component becomes less than 50 mass % (the proportion of the core resin component becomes more than 50 mass %), the recoverability rate after one day of compression becomes 70% or more, and such a case is preferable.

In the manner as described above, the nonwoven fabric 10 is prepared. In the place between the projection 122 of the support male material 120 and the projection 131 of the support female material 130, the fibers in the fiber web 110 are arranged and oriented in thickness direction, and the connecting portions 3 are formed. At this time, the connecting portions 3 in which the fibers are oriented in thickness direction (vertical direction) are formed on the surface directed to any direction around the projection 121. Thus, the space portion 4 of the nonwoven fabric 10 surrounded by the four connecting portions 3 is formed. In addition thereto, the outer surface fiber layer 2 on the second surface side Z2 in which the fibers are oriented in plane direction is formed between the top portion of the projection 121 and the bottom portion of the recess portion 132. Moreover, the outer surface fiber layer 1 on the first surface side Z1 in which the fibers are oriented in plane direction is formed between the bottom portion of the recess portion 122 and the top portion of the protruding portion 131.

In the nonwoven fabric 10 obtained, a surface on a lower side in FIG. 7(C) is the first surface side Z1, and a surface on a side opposite thereto serves as the second surface side Z2. That is, the first surface side Z1 in the nonwoven fabric 10 is a side on which the support male material 120 is arranged, and the second surface side Z2 is a side to which the first hot air W1 and the second hot air W2 are blown. Therefore, the number of fused points between the fibers in the outer surface fiber layer 2 on the second surface side Z2 becomes larger than the number of fused points between the fibers in the outer surface fiber layer 1 on the first surface side Z1 from a difference in an amount of blowing the first hot air W1. Further, the plane of the outer surface fiber layer 1 on the first surface side Z1 has a less rough-surface feeling and better texture than the plane of the outer surface fiber layer 2 on the second surface side Z2 due to the difference in heat quantity. Even if the step of blowing the first hot air W1 is omitted, the same effect is obtained by proximity to the second hot air W2. Furthermore, the fibers on a side of the support female material 130 (fibers serving as the outer surface fiber layer 2 on the second surface side Z2 in the nonwoven fabric 10) are pulled by fitting the supports into each other towards the support male material 120. Therefore, the fiber amount in the outer surface fiber layer 2 on the second surface side Z2 shaped in the top portion of the projection 121 of the support male material 120 becomes smaller than the fiber amount in the outer surface fiber layer 1 on the first surface side Z1 shaped in the bottom portion of the recess portion 122 of the support male material 120.

In the production method of the embodiment, thickness of the nonwoven fabric 10 is appropriately determined depending on the height of the projection 121 of the support male material 120 and the height of the projection 131 of the support female material 130. For example, if the height of the projection is increased, the apparent thickness of the sheet is increased, and if the height is decreased, the apparent thickness of the sheet is decreased. Moreover, if the height of the projection is increased, a fiber density of the nonwoven fabric 10 is decreased, and if the height is decreased, the nonwoven fabric 10 of the sheet is increased.

The nonwoven fabric of the present invention can be used in a variety of ways. For example, the nonwoven fabric can be suitably used as the topsheet of the absorbent article such as a disposable diaper for an adult or for an infant, a sanitary napkin, a panty liner, a urine pad and the like. Further, the nonwoven fabric is excellent in deformation characteristics during pressing force, and therefore can be also used in the form of a sublayer to be interposed between a topsheet and an absorbent body of a diaper, a sanitary item or the like, a covering sheet (core-wrapping sheet) of the absorbent body, or the like. Specific examples also include an embodiment in which the fabric is used as a top sheet, a gather, an exterior sheet and a wing of the absorbent articles. Further, specific examples also include an aspect of using the fabric in the form of a wipes sheet, a cleaning sheet, a filter, and a covering sheet of a thermal instrument.

With regard to the above embodiments, the present invention further discloses nonwoven fabrics described below.

<1>

A nonwoven fabric, comprising thermoplastic fibers, a first surface side and a second surface side which is a surface side opposite to the first surface side, wherein

the nonwoven fabric has outer surface fiber layers on the first surface side and the second surface side in which fibers are oriented in plane direction; and a plurality of connecting portions arranged between the outer surface fiber layer on the first surface side and the outer surface fiber layer on the second surface side, in which fibers are oriented in thickness direction of the nonwoven fabric; and

part of the fibers is fused to each other between the outer surface fiber layer on the first surface side, the outer surface fiber layer on the second surface side, and the connecting portions.

<2>

The nonwoven fabric according to the above item <1>, comprising a space portion surrounded by the connecting portions.

<3>

The nonwoven fabric according to the above item <2>, wherein an area proportion of the space portion of the nonwoven fabric in one surface is 5% or more and 90% or less, preferably 10% or more, and more preferably 15% or more; and preferably 80% or less, and more preferably 70% or less.

<4>

The nonwoven fabric according to the above item <2> or <3>, wherein a length of the connecting portion in plane direction is smaller than lengths of the outer surface fiber layer on the first surface side and the outer surface fiber layer on the second surface side in plane direction, in a cross section of the nonwoven fabric in thickness direction and in a cross section passing through a center of the space portion.

<5>

The nonwoven fabric according to the above item <4>, wherein a ratio of the length of the connecting portion in plane direction to the length of the outer surface fiber layer on the first surface side in plane direction is more than 0 and 0.9 or less, preferably 0.75 or less, and more preferably 0.5 or less; and preferably 0.001 or more, and more preferably 0.01 or more, in the cross section.

<6>

The nonwoven fabric according to the above item <4>, wherein a ratio of the length of the connecting portion in plane direction to the length of the outer surface fiber layer on the first surface side in plane direction is 0.01 or more and 0.5 or less, in the cross section.

<7>

The nonwoven fabric according to any one of the above items <4> to <6>, wherein a ratio of the length of the connecting portion in plane direction to the length of the outer surface fiber layer on the second surface side in plane direction is more than 0 and 0.9 or less, preferably 0.75 or less, and more preferably 0.5 or less; and preferably 0.001 or more, and more preferably 0.01 or more, in the cross section.

<8>

The nonwoven fabric according to any one of the above items <4> to <6>, wherein a ratio of the length of the connecting portion in plane direction to the length of the outer surface fiber layer on the second surface side in plane direction is 0.01 or more and 0.5 or less, in the cross section.

<9>

The nonwoven fabric according to any one of the above items <1> to

<8>, wherein the connecting portion has wall surfaces having a height in thickness direction of the nonwoven fabric and a width in plane direction of the nonwoven fabric along the extending direction of the outer surface fiber layer on the first surface side and the outer surface fiber layer on the second surface side, and the wall surfaces are arranged along a plurality of crossing different directions in plane view in the nonwoven fabric.
<10>

The nonwoven fabric according to any one of the above items <2> to <9>, wherein, among parts in at least four directions surrounding the space portion in the connecting portions, a difference between the lengths in plane direction is 2 mm or less, preferably 1 mm or less, and more preferably 0 (zero) mm, in a cross section of the nonwoven fabric in thickness direction and a cross section passing through a center of the space portion.

<11>

The nonwoven fabric according to any one of the above items <1> to <10>, wherein a plurality of the connecting portion are separated from each other and arranged in plane direction of the nonwoven fabric.

<12>

The nonwoven fabric according to any one of the above items <1> to <11>, comprising a plurality of the outer surface fiber layers in each of the first surface side and the second surface side of the nonwoven fabric, wherein the plurality of the outer surface fiber layers is separated from each other and arranged.

<13>

The nonwoven fabric according to the above item <12>, comprising a concavo-convex shape by separation arrangement of the outer surface fiber layer.

<14>

The nonwoven fabric according to any one of the above items <1> to <13>, wherein the outer surface fiber layer in the first surface side comprises two kinds having a length extending along each of crossing different directions in a plane view of the nonwoven fabric.

<15>

The nonwoven fabric according to the above item <14>, wherein one outer surface fiber layer of the two kinds of outer surface fiber layers is continuously extended in a longitudinal direction in a plane view of the nonwoven fabric, and a plurality of the one outer surface fiber layers are separated from each other and arranged relative to a crosswise direction perpendicular to the longitudinal direction.

<16>

The nonwoven fabric according to the above item <14> or <15>, wherein the other outer surface fiber layer of the two kinds of outer surface fiber layers is extended in the crosswise direction and is arranged by linking the one outer surface fiber layers, in a plane view of the nonwoven fabric.

<17>

The nonwoven fabric according to the above item <16>, wherein, on the first surface, a position of in the other outer surface fiber layer is formed to be lower than a position of the one outer surface fiber layer.

<18>

The nonwoven fabric according to the above item <16> or <17>, wherein a width of the other outer surface fiber layer in the longitudinal direction of the nonwoven fabric is formed to be narrower than a width of the one outer surface fiber layer in the crosswise direction of the nonwoven fabric.

<19>

The nonwoven fabric according to any one of the above items <14> to <18>, wherein the outer surface fiber layer on the second surface side covers separation space between the outer surface fiber layers on the first surface side, and a plurality of the outer surface fiber layer on the second surface side are separated from each other and arranged in rows along the longitudinal direction of the nonwoven fabric being extending direction of the outer surface fiber layer on the first surface side.

<20>

The nonwoven fabric according to the above item <19>, wherein a plurality of the longitudinal rows of the outer surface fiber layers on the second surface side is separated from each other and arranged in the crosswise direction perpendicular to the longitudinal direction.

<21>

The nonwoven fabric according to any one of the above items <1> to <20>, wherein the connecting portion links end portions of the outer surface fiber layer on the first surface side and the outer surface fiber layer on the second surface side.

<22>

The nonwoven fabric according to any one of the above items <1> to <21>, wherein, between the outer surface fiber layer on the first surface side and the outer surface fiber layer on the second surface side, a fiber amount in one side is adjusted to be smaller than a fiber amount in the other side.

<23>

The nonwoven fabric according to the above item <22>, wherein the fiber amount in the outer surface fiber layer on the first surface side is 1.1 times or more and 20 times or less, preferably 1.5 times or more, and more preferably 2 times or more; and preferably 10 times or less, and more preferably 5 times or less, as many as the fiber amount in the outer surface fiber layer on the second surface side.

<24>

The nonwoven fabric according to the above item <22>, wherein the fiber amount in the outer surface fiber layer on the first surface side is 2 times or more and 5 times or less as many as the fiber amount in the outer surface fiber layer on the second surface side.

<25>

The nonwoven fabric according to any one of the above items <1> to <24>, wherein the fibers being oriented in plane direction with regard to the outer surface fiber layer on the first surface side and the outer surface fiber layer on the second surface side of the nonwoven fabric means that a fiber vertical orientation ratio in the cross section in thickness direction of each of the outer surface fiber layer is less than 45%.

<26>

The nonwoven fabric according to any one of the above items <1> to <24>, wherein a fiber vertical orientation ratio in the cross section in thickness direction of the outer surface fiber layer on the first surface side and the outer surface fiber layer on the second surface side of the nonwoven fabric is 0% or more and less than 40%, and preferably 30% or more; and preferably 38% or less, and more preferably 37% or less.

<27>

The nonwoven fabric according to any one of the above items <1> to <24>, wherein a fiber vertical orientation ratio in the cross section in thickness direction of each of the outer surface fiber layer on the first surface side and the outer surface fiber layer on the second surface side of the nonwoven fabric is 30% or more and 37% or less.

<28>

The nonwoven fabric according to any one of the above items <1> to <27>, wherein the fibers of the connecting portion being oriented in thickness direction means that the fiber vertical orientation ratio in the cross section in thickness direction of the connecting portion is 60% or more.

<29>

The nonwoven fabric according to any one of the above items <1> to <27>, wherein a fiber vertical orientation ratio in the cross section in thickness direction of the connecting portion is 63% or more and 90% or less, preferably 65% or more, and more preferably 68% or more; and preferably 85% or less, and more preferably 80% or less.

<30>

The nonwoven fabric according to any one of the above items <1> to <27>, wherein a fiber vertical orientation ratio in the cross section in thickness direction of the connecting portion is 68% or more and 80% or less.

<31>

The nonwoven fabric according to any one of the above items <1> to <30>, wherein an apparent thickness of the nonwoven fabric is 1.5 mm or more and 10 mm or less, preferably 2 mm or more, and more preferably 3 mm or more; and preferably 9 mm or less, and more preferably 8 mm or less.

<32>

The nonwoven fabric according to any one of the above items <1> to <30>, wherein an apparent thickness of the nonwoven fabric is 3 mm or more and 8 mm or less.

<33>

The nonwoven fabric according to any one of the above items <1> to <32>, wherein a basis weight of the nonwoven fabric as a whole is 8 g/m² or more and 100 g/m² or less, preferably 60 g/m² or less, and more preferably 40 g/m² or less; and preferably 10 g/m² or more, and more preferably 15 g/m² or more.

<34>

An absorbent article comprising the nonwoven fabric according to any one of the above items <1> to <33>.

<35>

An absorbent article, wherein the first surface side of the nonwoven fabric according to any one of the above items <1> to <33> being a surface opposite to a surface to which hot air is blown during production is arranged toward a wearer's skin surface side as a topsheet.

<36>

An absorbent article, wherein the second surface side of the nonwoven fabric according to any one of the above items <1> to <33> being a surface to which hot air is blown during production is arranged toward a wearer's skin surface side as a topsheet.

<37>

A method for producing a nonwoven fabric, comprising a step of placing a fiber web on a support male material, which has a plurality of projections and a recess portion arranged between a plurality of the projections; and pushing and interposing the fiber web with the support male material, which has a recess portion and a projection corresponding to the projections and the recess portion of the support male material, from above the fiber web to shape the fiber web.

<38>

The method for producing a nonwoven fabric according to the above item

<37>, comprising a step of blowing hot air in a state in which the support male material and the support female material are fitted into each other by interposing the fiber web, wherein a bottom portion of the recess portion of the support male material and the support female material is formed into a structure through which hot air is blown.

### EXAMPLES

Hereinafter, the present invention will be described more in detail with reference to Examples, but the present invention is not limited thereto. Further, both terms “part” and “%” in the Examples are based on mass unless otherwise noted. The item “-” in the following Table means that there are no values corresponding to the item and the like.

### Example 1

A nonwoven fabric shown in FIG. 1 was prepared by using thermoplastic fibers of a sheath-core type (polyethylene terephthalate (PET) (core):polyethylene (PE) (sheath)=5:5 (mass ratio)) and having a fiber diameter of 1.8 dtex according to an air-through production method including a step shown in FIG. 7. The resultant material was taken as a nonwoven fabric sample in Example 1. Blowing treatment by first hot air W1 was applied thereto at a temperature of 160° C., an air speed of 54 m/s and a blowing time of 6 s. Blowing treatment by second hot air was applied thereto at a temperature of 160° C., an air speed of 6 m/s and a blowing time of 6 s.

The nonwoven fabric sample in Example 1 had an outer surface fiber layer 1 on a first surface side Z1, an outer surface fiber layer 2 on a second surface side Z2 and connecting portions 3, meeting the above-mentioned definition.

A length T1 of a connecting portion 31 was formed to be smaller than a length T2 of a first outer surface fiber layer 11 and a length T3 of the outer surface fiber layer 2. Moreover, a length T4 of a connecting portion 32 was formed to be smaller than a length T5 of a second outer surface fiber layer 12 and a length T6 of the outer surface fiber layer 2.

In the nonwoven fabric sample in Example 1, a fiber amount in the outer surface fiber layer 2 on the second surface side was adjusted to be smaller than a fiber amount in the outer surface fiber layer 1 on the first surface side.

### Example 2

A nonwoven fabric sample in Example 2 was prepared according to the method of Example 1 except that the temperature of the air-through in the first step was adjusted to 145° C., and an air speed was adjusted to 40 m/s.

The nonwoven fabric sample in Example 2 had an outer surface fiber layer 1 on a first surface side Z1, an outer surface fiber layer 2 on a second surface side Z2 and connecting portions 3, meeting the above-mentioned definition, and lengths T1 and T4 of the connecting portions were formed to be smaller than lengths T2 and T5 of the outer surface fiber layer 1 and lengths T3 and T6 of the outer surface fiber layer 2, respectively. Moreover, in the nonwoven fabric sample in Example 2, a fiber amount in the outer surface fiber layer 2 was adjusted to be smaller than a fiber amount in the outer surface fiber layer 1.

### Example 3

A nonwoven fabric sample in Example 3 was prepared according to the method of Example 1 except that thermoplastic fibers were of a sheath-core type (polyethylene terephthalate (PET) (core):polyethylene (PE) (sheath)=7:3 (mass ratio)) and having a fiber diameter of 3.2 dtex.

The nonwoven fabric sample in Example 3 had an outer surface fiber layer 1 on a first surface side Z1, an outer surface fiber layer 2 on a second surface side Z2 and connecting portions 3, meeting the above-mentioned definition, and lengths T1 and T4 of the connecting portions each were formed to be smaller than lengths T2 and T5 of the outer surface fiber layer 1 and lengths T3 and T6 of the outer surface fiber layer 2, respectively. Moreover, in the nonwoven fabric sample in Example 3, a fiber amount of the outer surface fiber layer 2 was formed to be smaller than a fiber amount in the outer surface fiber layer 1.

### Comparative Example 1

A concavo-convex nonwoven fabric having a shape shown in FIG. 1 of Patent Literature 3 described above was prepared by using thermoplastic fibers having a fiber diameter of 1.8 dtex according to an air-through production method including a production step described in the paragraph [0031] of the specification of the same literature. The resultant material was taken as a nonwoven fabric sample in Comparative Example 1. Blowing treatment by first hot air W1 was applied thereto at a temperature of 160° C., an air speed of 54 m/s and a blowing time of 3 s. Blowing treatment by second hot air was applied thereto at a temperature of 160° C., an air speed of 6 m/s and a blowing time of 3 s.

In the nonwoven fabric sample in Comparative Example 1, both a first projecting portion on a first surface side and a second projecting portion on a second surface side had a truncated cone shape or a hemispherical shape rounded in a top portion. The first projecting portion on the first surface side, the second projecting portion on the second surface side and an annular wall portion interposed between the first projecting portion and the second projecting portion were measured by applying mutatis mutandis the above-mentioned methods (Measurement of fiber vertical orientation ratio in outer surface fiber layers 1, 2 and connecting portions 3). This showed that the wall portion in the nonwoven fabric sample in Comparative Example 1 was not “a connecting portion in which fibers were oriented in thickness direction” in the nonwoven fabric according to the present invention.

Moreover, it was found that a length of the connecting portion was larger than lengths of outer surface fiber layers, respectively, and a top portion was rounded to have a concavo-convex shape gently toward the second outer surface fiber layer.

### Comparative Example 2

A flat nonwoven fabric shaped not to have a concavo-convex shape was prepared by using thermoplastic fibers having a fiber diameter of 1.8 dtex using an air-through production method, and the resultant material was taken as a nonwoven fabric sample in Comparative Example 2. The nonwoven fabric sample was a flat nonwoven fabric, and therefore there was no boundary specifying an outer surface fiber layer by the concavo-convex shape, and T1 to T6 were unable to be specified. A fiber orientation ratio in the outer surface fiber layer observed from an upper surface was measured.

### Comparative Example 3

A flat nonwoven fabric used in a surface material of Merries Pants L size (manufactured by Kao Corporation, 2016) was peeled, and taken as a nonwoven fabric sample in Comparative Example 3. The nonwoven fabric sample was a flat nonwoven fabric, and therefore there was no boundary specifying an outer surface fiber layer by the concavo-convex shape, and T1 to T6 were unable to be specified. A fiber orientation ratio in the outer surface fiber layer observed from an upper surface was measured.

### Comparative Example 4

A concavo-convex nonwoven fabric used in a surface material of Merries M size (manufactured by Kao Corporation, 2016) was peeled, and taken as a nonwoven fabric sample in Comparative Example 4. The nonwoven fabric sample was a concavo-convex nonwoven fabric, but an outer surface fiber layer 2 on a non-skin surface side (second surface side Z2) was flat, and therefore T1 to T6 were unable to be specified. A fiber orientation ratio of the fibers from a concavo-convex surface toward a flat surface was measured.

The following tests (1) to (4) were conducted on the Examples and Comparative Examples described above. Moreover, the following test (5) was also conducted on the Examples described above.

### (1) Compression Energy (WC), Compression Recovery Rate (RC)

In order to measure thickness recoverability and a deformation amount, compressive characteristics by compressional load up to 5 kPa in a normal mode were evaluated on a nonwoven fabric by using KES Compression Tester (KES FB-3, manufactured by Kato Tech Co., Ltd.) except that a speed of a terminal was set to 0.1 mm/s, and WC and RC indicated were read. As measured values, measurement was carried out on 3 points in the nonwoven fabric, and the measured values were averaged, and such operation was performed three times, and average values were taken as a WC value and an RC value.

The above-described WC value represents energy required for compression per unit area, and as the WC value is larger, the nonwoven fabric is further easily compressed.

The above-described RC value was expressed by percentage of a ratio of restored energy relative to energy during compression, and a larger RC value is judged to have better restoration properties against compression and have elastic properties.

(2) Compressive Deformation Amount (Compressive Deformation Amount Under Load of 0.1 to 2.5 kPa; Mm)

In the section (1), a deformation amount of 0.1 to 2.5 kPa was extracted and taken as a measured value. A higher value indicates that the nonwoven fabric is further significantly compressed relative to a load at which a person touches the nonwoven fabric. Accordingly as this value is larger, there is more compression, and the nonwoven fabric has greater cushioning properties. More specifically, a larger level of this numerical value indicates that the nonwoven fabric is harder to flatten in a compression direction by a small load, that is, the nonwoven fabric has higher shape-retaining properties, and has good elasticity. Moreover, the larger the numerical value indicates that the nonwoven fabric is easily flattened during a load of 2.5 kPa, and if the numerical value is large, the nonwoven fabric is significantly deformed upon touching the nonwoven fabric, and therefore the cushioning properties are easily felt.

### (3) Buckling Distortion

From a stress-strain curve of a compressive deformation amount by measurement using KES Compression Tester, an inflection point was sought and taken as a buckling load. A case where the inflection point was found was taken as A: presence of buckling distortion. A case where the inflection point was not found was taken as B: absence of buckling distortion. The nonwoven fabric having the buckling distortion has good elasticity.

### (4) Texture

The flat nonwoven fabric in Comparative Example 3 was rated as 3 points, and the concavo-convex nonwoven fabric in Comparative Example 4 was rated as 4 points, and based on 10 points, three researchers (twenties to thirties in their age) engaged in research and development of texture of the nonwoven fabric were asked to assume the best texture material in clothes and nonwoven fabrics which have been so far touched, and evaluation in 10 grades was conducted, and the evaluated values were averaged, and summarized in an integer. In assumption of touching a surface material of a diaper, the researchers each were asked to touch on a surface of a sample placed on a plane surface with a dominant hand. The evaluation was conducted directly by visual observation.

(5) Recoverability after One Day Compression

A nonwoven fabric was sandwiched between two acrylic plates together with a washer having thickness of 0.7 mm, a weight (20 kg) was placed thereon, and a load was applied to compress the nonwoven fabric to thickness of 0.7 mm. After standing for one day in this state, the weight and the acrylic plates were removed from the nonwoven fabric, and after 10 minutes an apparent thickness of the nonwoven fabric was measured. From this measurement value and an apparent thickness of the nonwoven fabric before compression (previously measured), recovery rate of thickness of the nonwoven fabric was calculated to evaluate the recoverability after one day compression of the nonwoven fabric.

TABLE 1

| | | Example 1 | Example 2 | Example 3 |
|---|---|---|---|---|
| Basis weight | g/m² | 32 | 33 | 30 |
| Apparent thickness | mm | 4.6 | 4.3 | 6.6 |
| Length of T1 | mm | 1.10 | 0.58 | 0.82 |
| Length of T2 | mm | 1.20 | 1.00 | 1.10 |
| Length of T3 | mm | 2.40 | 2.10 | 2.00 |
| Length of T4 | mm | 0.53 | 1.70 | 0.71 |
| Length of T5 | mm | 1.20 | 2.10 | 1.20 |
| Length of T6 | mm | 2.80 | 1.80 | 1.40 |
| Space ratio | % | 23.0 | 20.0 | 23.0 |
| Vertical orientation ratio in connecting portion | % | 72 | 66 | 63 |
| Vertical orientation ratio in outer surface fiber layers | % | 37 | 35 | 39 |
| on top surface side and back surface side | | | | |
| Fibre amount of outer surface fiber layer | g/m² | 22 | 23 | 24 |
| on first surface side | | | | |
| Fibre amount of outer surface fiber layer | g/m² | 5 | 5 | 6 |
| on second surface side | | | | |
| Area ratio in surface | % | 23 | 20 | 23 |
| on first surface side of space portion | | | | |
| Compression energy WC | N-m/m² | 1.14 | 0.87 | 1.40 |
| Compression recovery rate RC | % | 43 | 43 | 47 |
| Compressive deformation amount | mm | 3.9 | 2.7 | 5.3 |
| Buckling distortion | — | A | A | A |
| Texture | point | 8 | 7 | 8 |
| Recovery rate after one day compression | % | 60 | 60 | 72 |

| | | Comparative | Comparative | Comparative | Comparative |
|---|---|---|---|---|---|
| | | Example 1 | Example 2 | Example 3 | Example 4 |
| Basis weight | g/m² | 33 | 98 | 24 | 44 |
| Apparent thickness | mm | 4.0 | 6.0 | 1.0 | 1.4 |
| Length of T1 | mm | 3.26 | — | — | — |
| Length of T2 | mm | 1.47 | (Contact all) | (Contact all) | — |
| Length of T3 | mm | 0.75 | (Contact all) | (Contact all) | (Contact all) |
| Length of T4 | mm | 1.64 | — | — | — |
| Length of T5 | mm | 0.89 | (Contact all) | (Contact all) | — |
| Length of T6 | mm | 1.51 | (Contact all) | (Contact all) | (Contact all) |
| Space ratio | % | 15.0 | 0.0 | 0.0 | 4.7 |
| Vertical orientation ratio in connecting portion | % | 52 | — | — | — |
| Vertical orientation ratio in outer surface fiber layers | % | 39 | 37 | 39 | 46 |
| on top surface side and back surface side | | | | | |
| Fibre amount of outer surface fiber layer | g/m² | — | — | — | — |
| on first surface side | | | | | |
| Fibre amount of outer surface fiber layer | g/m² | — | — | — | — |
| on second surface side | | | | | |
| Area ratio in surface | % | — | — | — | — |
| on first surface side of space portion | | | | | |
| Compression energy WC | N-m/m² | 1.00 | 0.95 | 0.09 | 0.20 |
| Compression recovery rate RC | % | 43 | 65 | 56 | 48 |
| Compressive deformation amount | mm | 2.1 | 1.8 | 0.4 | 0.6 |
| Buckling distortion | — | A | B | B | B |
| Texture | point | 5 | 1 | 3 | 4 |

Table 1 shows that compression energy (WC) is large in Examples 1 to 3 and Comparative Examples 1 and 2 in which apparent thickness is high, and such samples have excellent cushioning properties. Further, compression recovery rates (RC) are 40% or more for all, and all are excellent in thickness recovery rate. Moreover, orientation of the connecting portions was not in vertical orientation in the nonwoven fabric having a concavo-convex shape as in Comparative Example 1, and therefore the compressive deformation amount was smaller in comparison with Examples 1 and 2, and the texture was superior in Examples 1 to 3. Further, where the material was the flat nonwoven fabric and had an apparent thickness equivalent to or more than the level (as in Comparative Example 2), the fiber amount was large, and therefore the compressive deformation amount could not be increased, and the buckling distortion was not induced. Therefore the texture was superior in Examples 1 to 3.

That is, in Examples 1 to 3, the nonwoven fabric had good elasticity upon lightly touching the fabrics by the presence of the buckling distortion, and the compressive deformation amount was increased by the presence of the buckling distortion to exhibit good quality cushioning unable to be fully expressed only by the conventional compression energy (WC) in terms of KES. As a result, even though the nonwoven fabrics in Examples 1 to 3 had similar thickness in comparison with Comparative Examples 1 and 2, a superb cushion feeling by superb thickness recovery, good elasticity and a large compressive deformation amount was observed, and the texture was superior in the Examples.

Moreover, in Comparative Example 3 in which the fiber amount was small, thickness could not be produced, and the texture was superior in Examples 1 to 3. The concavo-convex nonwoven fabric in Comparative Example 4 had moderate thickness, but the second fiber layer was flat, and therefore the compressive deformation amount was larger in Examples 1 to 3.

As described above, in Examples 1 to 3, the connecting portions were vertically oriented with a plane-oriented part, and thus the apparent thickness was able to be realized with low basis weight. Moreover, vertically oriented parts formed posts to exhibit the buckling distortion, and the deformation amount (compressive deformation amount) to be felt by a person in the vicinity of the load was able to be increased in comparison with Comparative Examples 1 to 4. Thus, the texture was significantly improved.

Further, the buckling distortion was exhibited in Examples 1 to 3, and thus good elasticity was felt upon rubbing the nonwoven fabric with a finger (small load less than 100 Pa), and the nonwoven fabrics had satisfactory texture at which a comfortable soft and thick feeling was able to be felt. Moreover, in Examples 1 to 3, partial compression in the vicinity of a force point of the nonwoven fabric was induced upon pushing the nonwoven fabric with the finger (for example, pressing force of about 2.5 kPa), and spreading of deformation from the force point to a circumference was limited in comparison with Comparative Examples.

Furthermore, among Examples 1 to 3, the nonwoven fabric in Example 3 in which a mass ratio of PE (temperature of glass transition component is lower in comparison with PET being core resin) being sheath resin was decreased had excellent recoverability after one day compression, and it was found that the nonwoven fabric in Example 3 had high thickness recoverability even after the nonwoven fabric is compressed with packing and the like.

Having described our invention as related to these embodiments and Examples, it is our intention that the invention not be limited by any of the details of the description, unless otherwise specified, but rather be construed broadly within its spirit and scope as set out in the accompanying claims.

This application claims priority from Patent Application No. 2017-168000 filed in Japan on Aug. 31, 2017, which is incorporated herein by reference in its entirety.

### DESCRIPTION OF SYMBOLS

1 Outer surface fiber layer on first surface side
11 First outer surface fiber layer
12 Second outer surface fiber layer
2 Outer surface fiber layer on second surface side
3 Connecting portion
31 First connecting portion
32 Second connecting portion
39 End portion of connecting portion
10 Nonwoven fabric
Z1 First surface side
Z2 Second surface side

1. A nonwoven fabric, comprising thermoplastic fibers, a first surface side and a second surface side which is a surface side opposite to the first surface side, wherein
the nonwoven fabric has outer surface fiber layers on the first surface side and the second surface side in which fibers are oriented in plane direction; and a plurality of connecting portions arranged between the outer surface fiber layer on the first surface side and the outer surface fiber layer on the second surface side, in which fibers are oriented in thickness direction of the nonwoven fabric;
a fiber vertical orientation ratio in the cross section in thickness direction of the nonwoven fabric is 60% or more in the connecting portion; and
part of the fibers is fused to each other between the outer surface fiber layer on the first surface side, the outer surface fiber layer on the second surface side, and the connecting portions.

2. A nonwoven fabric, comprising thermoplastic fibers, a first surface side and a second surface side which is a surface side opposite to the first surface side, wherein
the nonwoven fabric has outer surface fiber layers on the first surface side and the second surface side in which fibers are oriented in plane direction; and a plurality of connecting portions arranged between the outer surface fiber layer on the first surface side and the outer surface fiber layer on the second surface side, in which fibers are oriented in thickness direction of the nonwoven fabric;
part of the fibers is fused to each other between the outer surface fiber layer on the first surface side, the outer surface fiber layer on the second surface side, and the connecting portions;
the connecting portion has wall surfaces having a height in thickness direction of the nonwoven fabric and a width in plane direction of the nonwoven fabric along the extending direction of the outer surface fiber layer on the first surface side and the outer surface fiber layer on the second surface side;
the connecting portion comprises two kinds of the connecting portions having the wall surfaces arranged along different directions crossing each other in plane view in the nonwoven fabric;
a fiber vertical orientation ratio in the cross section in thickness direction of the nonwoven fabric in the connecting portion along one direction is 60% or more; and
a fiber vertical orientation ratio in the cross section in thickness direction of the nonwoven fabric in the connecting portion along the other direction is 60% or more.

3. The nonwoven fabric according to claim 1, wherein the connecting portion has wall surfaces having a height in thickness direction of the nonwoven fabric and a width in plane direction of the nonwoven fabric along the extending direction of the outer surface fiber layer on the first surface side and the outer surface fiber layer on the second surface side, and the wall surfaces are arranged along a plurality of crossing different directions in plane view in the nonwoven fabric.

4. The nonwoven fabric according to claim 1, comprising a space portion surrounded by the connecting portions.

5. The nonwoven fabric according to claim 4, wherein a length of the connecting portion in plane direction is smaller than lengths of the outer surface fiber layer on the first surface side and the outer surface fiber layer on the second surface side in plane direction, in a cross section of the nonwoven fabric in thickness direction and in a cross section passing through a center of the space portion.

6. The nonwoven fabric according to claim 4,
wherein the connecting portion has wall surfaces having a height in thickness direction of the nonwoven fabric and a width in plane direction of the nonwoven fabric along the extending direction of the outer surface fiber layer on the first surface side and the outer surface fiber layer on the second surface side;
the connecting portion comprises two kinds of the connecting portions having the wall surfaces arranged along different directions crossing each other in plane view in the nonwoven fabric; and
in the connecting portions, a difference between the length of a cross section fiber layer of the connecting portion along one direction in plane direction and the length of the cross section fiber layer of the connecting portion along the other direction in plane direction is 2 mm or less, in a cross section of the nonwoven fabric in thickness direction and a cross section passing through a center of the space portion.

7. The nonwoven fabric according to claim 1, wherein a plurality of the connecting portion are separated from each other and arranged in plane direction of the nonwoven fabric.

8. The nonwoven fabric according to claim 1, comprising a plurality of the outer surface fiber layers in each one or both of the first surface side and the second surface side of the nonwoven fabric, wherein the plurality of the outer surface layers is separated from each other and arranged.

9. The nonwoven fabric according to claim 8, comprising a concavo-convex shape by separation arrangement of the outer surface fiber layer.

10. The nonwoven fabric according to claim 1, wherein the outer surface fiber layer in the first surface side comprises two kinds having a length extending along each of crossing different directions in a plane view of the nonwoven fabric.

11. The nonwoven fabric according to claim 10, wherein one outer surface fiber layer of the two kinds of the outer surface fiber layers is continuously extended in a longitudinal direction in a plane view of the nonwoven fabric, and a plurality of the one outer surface fiber layers is separated from each other and arranged relative to a crosswise direction perpendicular to the longitudinal direction.

12. The nonwoven fabric according to claim 11, wherein the other outer surface fiber layer of the two kinds of outer surface fiber layers is extended in the crosswise direction and is arranged by linking the one outer surface fiber layers, in a plane view of the nonwoven fabric.

13. The nonwoven fabric according to claim 12, wherein, on the first surface side, a position of the other outer surface fiber layer is formed to be lower than a position of the one outer surface fiber layer.

14. The nonwoven fabric according to claim 12, wherein a width of the other outer surface fiber layer in the longitudinal direction of the nonwoven fabric is formed to be narrower than a width of the one outer surface fiber layer in the crosswise direction of the nonwoven fabric.

15. The nonwoven fabric according to claim 10, wherein the outer surface fiber layer on the second surface side covers separation space between the outer surface fiber layers on the first surface side, and a plurality of the outer surface fiber layer on the second surface side is separated from each other and arranged in rows along the longitudinal direction of the nonwoven fabric being extending direction of the outer surface fiber layer on the first surface side.

16. The nonwoven fabric according to claim 15, wherein a plurality of the longitudinal rows of the outer surface fiber layers on the second surface side is separated from each other and arranged in the crosswise direction perpendicular to the longitudinal direction.

17. The nonwoven fabric according to claim 1, wherein the connecting portion links end portions of the outer surface fiber layer on the first surface side and the outer surface fiber layer on the second surface side.

18. The nonwoven fabric according to claim 1, wherein, between the outer surface fiber layer on the first surface side and the outer surface fiber layer on the second surface side, a fiber amount in one side is adjusted to be smaller than a fiber amount in the other side.

19. An absorbent article comprising the nonwoven fabric according to claim 1.

20. A method for producing a nonwoven fabric, comprising a step of placing a fiber web on a support male material, which has a plurality of projections and a recess portion arranged between a plurality of the projections; and pushing and interposing the fiber web with a support female material, which has a recess portion and a projection corresponding to the projections and the recess portion of the support male material, from above the fiber web to shape the fiber web.

21-38. (canceled)
